SEQUENCE LISTING (69 sequences)

SEQ ID NO: 1 - protein, 10 aa, synthetic construct
  REGION 1..10: note = Complementarity-determining regions (CDR)
GDSISNYYWS

SEQ ID NO: 2 - protein, 16 aa, synthetic construct
  REGION 1..16: note = Complementarity-determining regions (CDR)
YFYYSGSTNYNPSLKS

SEQ ID NO: 3 - protein, 7 aa, synthetic construct
  REGION 1..7: note = Complementarity-determining regions (CDR)
DRGWADY

SEQ ID NO: 4 - protein, 12 aa, synthetic construct
  REGION 1..12: note = Complementarity-determining regions (CDR)
RTSQSISSSYLA

SEQ ID NO: 5 - protein, 7 aa, synthetic construct
  REGION 1..7: note = Complementarity-determining regions (CDR)
GASSRAT

SEQ ID NO: 6 - protein, 8 aa, synthetic construct
  REGION 1..8: note = Complementarity-determining regions (CDR)
QQYGSSIT

SEQ ID NO: 7 - protein, 7 aa, synthetic construct
  REGION 1..7: note = Complementarity-determining regions (CDR)
SFNYYWS

SEQ ID NO: 8 - protein, 16 aa, synthetic construct
  REGION 1..16: note = Complementarity-determining regions (CDR)
YIYYSGSTYSNPSLKS

SEQ ID NO: 9 - protein, 9 aa, synthetic construct
  REGION 1..9: note = Complementarity-determining regions (CDR)
GYNWNYFDY

SEQ ID NO: 10 - protein, 11 aa, synthetic construct
  REGION 1..11: note = Complementarity-determining regions (CDR)
RASQSVDNNLV

SEQ ID NO: 11 - protein, 7 aa, synthetic construct
  REGION 1..7: note = Complementarity-determining regions (CDR)
GASTRAT

SEQ ID NO: 12 - protein, 10 aa, synthetic construct [sequence omitted]

SEQ ID NO: 13 - protein, 12 aa, synthetic construct
  REGION 1..12: note = Complementarity-determining regions (CDR)
GFSLSAGGVGVG

SEQ ID NO: 14 - protein, 15 aa, synthetic construct
  REGION 1..15: note = Complementarity-determining regions (CDR)
LIYWNDDKRYSPSLR

SEQ ID NO: 15 - protein, 13 aa, synthetic construct
  REGION 1..13: note = Complementarity-determining regions (CDR)
SHYDYDWGSYFDY

SEQ ID NO: 16 - protein, 17 aa, synthetic construct
  REGION 1..17: note = Complementarity-determining regions (CDR)
RSSQSLLDSDDGNTYLD

SEQ ID NO: 17 - protein, 7 aa, synthetic construct
  REGION 1..7: note = Complementarity-determining regions (CDR)
TLSYRAS

SEQ ID NO: 18 - protein, 9 aa, synthetic construct
  REGION 1..9: note = Complementarity-determining regions (CDR)
MQRIEFPIT

SEQ ID NO: 19 - protein, 12 aa, synthetic construct
  REGION 1..12: note = Complementarity-determining regions (CDR)
GGSISSANYYWT

SEQ ID NO: 20 - protein, 16 aa, synthetic construct
  REGION 1..16: note = Complementarity-determining regions (CDR)
YIYYSGSTYCNPSLKS

SEQ ID NO: 21 - protein, 9 aa, synthetic construct
  REGION 1..9: note = Complementarity-determining regions (CDR)
GYNWNYFDY

SEQ ID NO: 22 - protein, 11 aa, synthetic construct
  REGION 1..11: note = Complementarity-determining regions (CDR)
RASQSVSSNLA

SEQ ID NO: 23 - protein, 7 aa, synthetic construct
  REGION 1..7: note = Complementarity-determining regions (CDR)
GASTRAT

SEQ ID NO: 24 - protein, 10 aa, synthetic construct
  REGION 1..10: note = Complementarity-determining regions (CDR)
QQYNKWPPWT

SEQ ID NO: 25 - protein, 10 aa, synthetic construct
  REGION 1..10: note = Complementarity-determining regions (CDR)
GFAFSSYGMH

SEQ ID NO: 26 - protein, 17 aa, synthetic construct
  REGION 1..17: note = Complementarity-determining regions (CDR)
VISYDGNNKYYADSVKG

SEQ ID NO: 27 - protein, 18 aa, synthetic construct
  REGION 1..18: note = Complementarity-determining regions (CDR)
DLVVRGIRGYYYYFGMDV

SEQ ID NO: 28 - protein, 16 aa, synthetic construct
  REGION 1..16: note = Complementarity-determining regions (CDR)
RSSQSLLHSNGYNYLD

SEQ ID NO: 29 - protein, 7 aa, synthetic construct
  REGION 1..7: note = Complementarity-determining regions (CDR)
LGSNRAS

SEQ ID NO: 30 - protein, 9 aa, synthetic construct
  REGION 1..9: note = Complementarity-determining regions (CDR)
MQGLQTPIT

SEQ ID NO: 31 - protein, 10 aa, synthetic construct
  REGION 1..10: note = Complementarity-determining regions (CDR)
GFTFSNYGIH

SEQ ID NO: 32 - protein, 17 aa, synthetic construct
  REGION 1..17: note = Complementarity-determining regions (CDR)
VIWFDGRNKYYADSVKG

SEQ ID NO: 33 - protein, 12 aa, synthetic construct
  REGION 1..12: note = Complementarity-determining regions (CDR)
DPFDYGDSFFDY

SEQ ID NO: 34 - protein, 11 aa, synthetic construct
  REGION 1..11: note = Complementarity-determining regions (CDR)
RASQDISNYLA

SEQ ID NO: 35 - protein, 6 aa, synthetic construct
  REGION 1..6: note = Complementarity-determining regions (CDR)
AASTLQ

SEQ ID NO: 36 - protein, 9 aa, synthetic construct
  REGION 1..9: note = Complementarity-determining regions (CDR)
QKYNSAPLT

SEQ ID NO: 37 - protein, 10 aa, synthetic construct
  REGION 1..10: note = Complementarity-determining regions (CDR)
GFTFSDYYMT

SEQ ID NO: 38 - protein, 17 aa, synthetic construct
  REGION 1..17: note = Complementarity-determining regions (CDR)
YISISGSITHYADSVKG

SEQ ID NO: 39 - protein, 12 aa, synthetic construct
  REGION 1..12: note = Complementarity-determining regions (CDR)
DGAAAGTDAFDI

SEQ ID NO: 40 - protein, 11 aa, synthetic construct
  REGION 1..11: note = Complementarity-determining regions (CDR)
RASQNVSSNLA

SEQ ID NO: 41 - protein, 7 aa, synthetic construct
  REGION 1..7: note = Complementarity-determining regions (CDR)
GASTRAT

SEQ ID NO: 42 - protein, 8 aa, synthetic construct
  REGION 1..8: note = Complementarity-determining regions (CDR)
QQYHYWPT

SEQ ID NO: 43 - protein, 10 aa, synthetic construct
  REGION 1..10: note = Complementarity-determining regions (CDR)
GYTFTGFYMH

SEQ ID NO: 44 - protein, 17 aa, synthetic construct
  REGION 1..17: note = Complementarity-determining regions (CDR)
WINPNSGGTYYVQKFQG

SEQ ID NO: 45 - protein, 12 aa, synthetic construct
  REGION 1..12: note = Complementarity-determining regions (CDR)
DGYSSGEDWFDP

SEQ ID NO: 46 - protein, 16 aa, synthetic construct
  REGION 1..16: note = Complementarity-determining regions (CDR)
KSSQSLLHSGGKTYLY

SEQ ID NO: 47 - protein, 7 aa, synthetic construct
  REGION 1..7: note = Complementarity-determining regions (CDR)
EVSNRFS

SEQ ID NO: 48 - protein, 9 aa, synthetic construct
  REGION 1..9: note = Complementarity-determining regions (CDR)
MQSIHLPLT

SEQ ID NO: 49 - protein, 230 aa, synthetic construct
  REGION 1..230: note = An exemplary human TREM-2 amino acid sequence
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 50 - protein, 470 aa, synthetic construct
  REGION 1..470: note = exemplary GPNMB (Transmembrane glycoprotein NMB ) amino acidsequence
KRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPAS

SEQ ID NO: 51 - protein, 7 aa, synthetic construct
  REGION 1..7: note = Complementarity-determining regions (CDR)
FSNYYMA

SEQ ID NO: 52 - protein, 10 aa, synthetic construct
  REGION 1..10: note = Complementarity-determining regions (CDR)
SLTNSGGSTY

SEQ ID NO: 53 - protein, 7 aa, synthetic construct
  REGION 1..7: note = Complementarity-determining regions (CDR)
EWAGSGY

SEQ ID NO: 54 - protein, 7 aa, synthetic construct
  REGION 1..7: note = Complementarity-determining regions (CDR)
NVGNNLA

SEQ ID NO: 55 - protein, 7 aa, synthetic construct
  REGION 1..7: note = Complementarity-determining regions (CDR)
YTSNRFT

SEQ ID NO: 56 - protein, 7 aa, synthetic construct
  REGION 1..7: note = Complementarity-determining regions (CDR)
RTYNSPW

SEQ ID NO: 57 - DNA, 36 bp, synthetic construct
  misc_feature 1..36: note = T7 RNA polymerase promoter sequence
  source 1..36: mol_type = other DNA
scgattgaggccggtaatacgactcactataggggc

SEQ ID NO: 58 - DNA, 18 bp, synthetic construct
  misc_feature 1..18: note = Single strand DNA oligonucleotide
  source 1..18: mol_type = other DNA
ggagggggttgaggtgtt

SEQ ID NO: 59 - DNA, 23 bp, synthetic construct
  misc_feature 1..23: note = Single strand DNA oligonucleotide
  source 1..23: mol_type = other DNA
tgtgcacttttattggtctcaag

SEQ ID NO: 60 - DNA, 20 bp, synthetic construct
  misc_feature 1..20: note = Single strand DNA oligonucleotide
  source 1..20: mol_type = other DNA
attatcggagcgcctttctc

SEQ ID NO: 61 - DNA, 20 bp, synthetic construct
  misc_feature 1..20: note = Single strand DNA oligonucleotide
  source 1..20: mol_type = other DNA
agcagaccagctttcctcag

SEQ ID NO: 62 - DNA, 19 bp, synthetic construct
  misc_feature 1..19: note = Single strand DNA oligonucleotide
  source 1..19: mol_type = other DNA
cccaagtggaacacagcac

SEQ ID NO: 63 - DNA, 20 bp, synthetic construct
  misc_feature 1..20: note = Single strand DNA oligonucleotide
  source 1..20: mol_type = other DNA
gatgctggctgcaagaaact

SEQ ID NO: 64 - DNA, 23 bp, synthetic construct
  misc_feature 1..23: note = Single strand DNA oligonucleotide
  source 1..23: mol_type = other DNA
ccctcacactcagatcatcttct

SEQ ID NO: 65 - DNA, 19 bp, synthetic construct
  misc_feature 1..19: note = Single strand DNA oligonucleotide
  source 1..19: mol_type = other DNA
gctacgacgtgggctacag

SEQ ID NO: 66 - DNA, 26 bp, synthetic construct
  misc_feature 1..26: note = Single strand DNA oligonucleotide
  source 1..26: mol_type = other DNA
aaaatcatccaaaagatactgaacaa

SEQ ID NO: 67 - DNA, 20 bp, synthetic construct
  misc_feature 1..20: note = Single strand DNA oligonucleotide
  source 1..20: mol_type = other DNA
ctttggttcttccgttgagg

SEQ ID NO: 68 - DNA, 18 bp, synthetic construct
  misc_feature 1..18: note = Single strand DNA oligonucleotide
  source 1..18: mol_type = other DNA
accctgcagtggttcgag

SEQ ID NO: 69 - DNA, 19 bp, synthetic construct
  misc_feature 1..19: note = Single strand DNA oligonucleotide
  source 1..19: mol_type = other DNA
cttgcacaaggaagcttgg

(19) United States
(12) Patent Application Publication
(10) Pub. No.: US 2022/0372145 A1
(43) Pub. Date: Nov. 24, 2022

(54) METHODS OF TREATING CANCER

(71) Applicant: Yeda Research and Development Co. Ltd., Rehovot (IL)

(72) Inventors: Ido AMIT, Rehovot (IL); Assaf WEINER, Rehovot (IL); Yonatan KATZENELENBOGEN, Rehovot (IL); Adam YALIN, Rehovot (IL); Fadi SHEBAN, Rehovot (IL)

(73) Assignee: Yeda Research and Development Co. Ltd., Rehovot (IL)

(21) Appl. No.: 17/876,641

(22) Filed: Jul. 29, 2022

Related U.S. Application Data
(63) Continuation of application No. PCT/IL2021/050102, filed on Jan. 28, 2021.

(30) Foreign Application Priority Data
Jan. 30, 2020 (IL) 272390

(51) Int. Cl. C07K 16/28; C07K 16/30

(52) U.S. Cl. CPC C07K 16/2803; C07K 16/30; C07K 2317/24; C07K 2317/31

(57) ABSTRACT

A method of reducing the immune suppressor activity of myeloid cells is disclosed. The method comprises contacting myeloid cells with an effective amount of an agent which specifically reduces the amount and/or activity of myeloid cells expressing both Triggering Receptor Expressed On Myeloid Cells 2 (Trem2) and Transmembrane glycoprotein NMB (Gpnmb). The method can be used for treating cancer. Antibodies and bi-specific antibodies are also disclosed.

### RELATED APPLICATIONS

This application is a Continuation of PCT Patent Application No. PCT/IL2021/050102 having International filing date of Jan. 28, 2021, which claims the benefit of priority of Israel Patent Application No. 272390 filed on Jan. 30, 2020. The contents of the above applications are all incorporated by reference as if fully set forth herein in their entirety.

### SEQUENCE LISTING STATEMENT

The XML file, entitled 93096SequenceListing.xml, created on Jul. 29, 2022 comprising 89,055 bytes, submitted concurrently with the filing of this application is incorporated herein by reference.

### FIELD AND BACKGROUND OF THE INVENTION

The present invention, in some embodiments thereof, relates to a method of treating cancer by reducing the immune suppressor activity of myeloid cells and, more particularly, but not exclusively, to solid cancers.

Many essential determinants of immune function cannot be precisely characterized by traditional surface markers, and it is unclear how the internal processing and integration of these signals translate toward immune activation, suppression and inflammation. Myeloid derived suppressor cells (MDSCs) are known to promote a suppressive environment for effector T cells within the tumor microenvironment (TME) and support tumor growth and immune dysfunction. Despite MDSC critical impact on treatment outcome in a broad spectrum of human disease and cancer types, their precise functional roles and molecular identity have been elusive and ill defined. MDSC do not conform to conventional surface-marker based classification schemes, and are classified using broad myeloid surface markers, various cellular assays and metabolic properties, including expression of an immune suppressive metabolic pathway expressing arginase 1 (Arg1). A thorough molecular understanding of this important and heterogeneous group of myeloid cells, based on their suppressive metabolic potential, may lead to identification of their molecular markers, pathways and activity—ultimately leading to more effective biomarkers and targeted immunotherapy.

Background art includes Kim et al., Cancers (Basel). 2019 September; 11(9): 1315; WO 2017/058866 and US Application No. 20180043014.

### SUMMARY OF THE INVENTION

According to an aspect of the present invention there is provided a method of reducing the immune suppressor activity of myeloid cells, the method comprising contacting myeloid cells with an effective amount of an agent which specifically reduces the amount and/or activity of myeloid cells expressing both Triggering Receptor Expressed On Myeloid Cells 2 (Trem2) and Transmembrane glycoprotein NMB (Gpnmb), thereby reducing the immune suppressor activity of myeloid cells.

According to an aspect of the present invention there is provided a method of treating cancer in a subject in need thereof, the method comprising administering to the subject an effective amount of an agent which specifically downregulates the amount and/or the activity of myeloid cells of the subject expressing both Triggering Receptor Expressed On Myeloid Cells 2 (Trem2) and Transmembrane glycoprotein NMB (Gpnmb), thereby treating the cancer.

According to an aspect of the present invention there is provided a method of treating cancer in a subject in need thereof, the method comprising:
(a) reducing the immune suppressor activity of myeloid cells according to the method of claim 3, wherein the myeloid cells are derived from the subject; and subsequently;
(b) transplanting the myeloid cells to the subject, thereby treating the cancer.

According to an aspect of the present invention there is provided a method of treating cancer in a subject in need thereof, the method comprising administering to the subject a therapeutically effective amount of
(i) a first agent which down-regulates the amount and/or activity of Trem2; and
(ii) a second agent which specifically down-regulates the amount and/or activity of Gpnmb, thereby treating the cancer.

According to an aspect of the present invention there is provided a bispecific antibody, comprising a first antigen-binding domain capable of specifically binding to Trem2 and a second antigen-binding domain capable of specifically binding to Gpnmb.

According to an aspect of the present invention there is provided a method of treating cancer in a subject comprising:
(a) analyzing in a sample of the subject for the presence of myeloid cells which express both Trem2 and Gpnmb; and
(b) when there is an amount of the myeloid cells above a predetermined amount, treating the subject with a therapeutically effective amount of an agent that targets Trem2 and/or Gpnmb; or when there is an amount of the cells below a predetermined amount treating the subject with a therapeutically effective amount of a chemotherapeutic agent other than the agent that targets Trem2 and/or Gpnmb.

According to some embodiments, the contacting is effected in vivo.

According to some embodiments, the contacting is effected ex vivo.

According to some embodiments, the agent comprises a bi-specific antibody.

According to some embodiments, a first target of the bi-specific antibody is Trem2 and a second target of the bi-specific antibody is Gpnmb.

According to some embodiments, the first agent and the second agent are inhibitory antibodies.

According to some embodiments, the bi-specific antibody is attached to a cytotoxic agent.

According to some embodiments, the cancer is a solid cancer.

According to some embodiments, the solid cancer is selected from the group consisting of lung cancer, liver cancer, ovarian cancer, gastric cancer and breast cancer.

According to some embodiments, the lung cancer is non-small cell lung cancer.

According to some embodiments, the lung cancer is small cell lung cancer.

According to some embodiments, the liver cancer is Hepatocellular carcinoma.

According to some embodiments, the method further comprises administering to the subject a therapeutically effective amount of a checkpoint inhibitor.

According to some embodiments, the method further comprises administering to the subject a therapeutically effective amount of a Brutons tyrosine kinase (Btk) inhibitor.

According to some embodiments, the Brutons tyrosine kinase (Btk) inhibitor is selected from the group consisting of ibrutinib, acalabrutinib and Spebrutinib.

According to some embodiments, the agent that targets Trem2 and/or Gpnmb antibody is an antibody.

According to some embodiments, the antibody is attached to a cytotoxic agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1: INs-seq: an integrated technology for scRNA-seq and intracellular protein measurements. A. Schematics of INs-seq experimental approach.

FIGS. 2A-G: Trem2 defines two populations of tumor infiltrating suppressive myeloid cells. A. Schematics of experimental design. B. Gene expression heatmap of 42 genes from 8580 cells clustered into 77 metacells of Arg1+ and Arg1− cells. Upper bar plot shows Arg1 enrichment score (fraction in the Arg1+ over Arg1− samples). C. Gene-gene Pearson correlation heatmap of 42 markers genes within the Arg1+ and Arg1− metacells. Upper bar plot shows Pearson correlation between gene expression and Arg1 enrichment score. D. qPCR analysis of Arg1 and Trem2 (E) expression fold change in the different cell populations compared to Ly6c+ F. Representative of flow cytometry plots of Pdpn versus Ly6C, Cx3cr1 and Gpnmb of cells isolated from MCA205 CD45+CD11b+ population. G. UMAP projection of CyToF data of MCA205 CD45+ immune cells. Detected protein levels of Arg1, Pdpn, Ly6c, Trem2 and Cx3cr1 is shown by color gradient as indicated in the plot.

FIGS. 3A-H: Trem2 promotes T cell dysfunction and tumor-immune escape. A. Two dimensional graph projection of 115 metacells representing 12081 myeloid cells. Different colors represent different cell population as indicated in the plot. B. Projection of key marker genes onto the graph plot. C. Scatterplot showing the mean UMI counts (log 2 scale) of Mregs (y axis) compared with monocytes (Ace) (x axis). D. Scatterplot showing the mean UMI counts (log 2 scale) of TAMs (y axis) compared with monocytes (Ace) (x axis). E. Heatmap showing enrichment of transcription factors binding sites in the regulatory regions for each cluster. Only TFs with significant Normalized Enrichment Score (NES>3.5) and mean expression above 0.1 UMI in the relevant cluster are shown. F. Percentage of key cell populations in WT and Trem2 KO in the MCA205 tumor microenvironment (day 19). Each point represents one animal; black line indicates average percentage. G. MCA205 tumor volume (day 19) of WT and Trem2 KO mice. Each point represents one animal, red line represents mean volume. Error bars indicate mean±SEM. (p=0.007, one-way ANOVA) H. Flow cytometry histogram for T cell proliferation analysis showing cell proliferation dye eFluor™ 450 fluorescent signal of WT splenic CD8 T cells stimulated with anti-CD3/anti-CD28 and co-cultured for 48 hours with MCA205 intra-tumoral CD11b+ Ccr2+ (Mon), Cx3cr1+ (TAM) or Gpnmb+ (Mreg) cells.

FIGS. 4A-F: Trem2 defines two populations of tumor infiltrating suppressive myeloid cells. A. FACS plot showing gating strategy for sorting CD45+ CD11b+ Arg1+ cells. B. qPCR values for Arg1 mRNA in INs-seq Arg1+ and Arg1− cells in the TME. C. Scatterplot showing the average UMI counts for selected genes (Arg1, Trem2, Cts1 and Plac8) in 77 metacells (y axis) compared with enrichment score (log 2 scale) in Arg1+ vs Arg1− cells (x axis). Scale bar indicates enrichment score D. Two dimensional graph projection of 77 metacells representing 8156 Arg1+ and Arg1− cells. Color indicates log 2 enrichment in Arg1+E. Bar plot showing the indicated genes expression (x axis) with correlation to Arg1 protein ratio F. UMAP projection of CyToF data of MCA205 CD45+ immune cells. Detected protein levels of the indicated proteins shown by color gradient as indicated in the plot.

FIGS. 5A-C: Trem2 promotes T cell dysfunction and tumor-immune escape. A. Two-dimensional graph projection of 82 metacells representing 15,946 intratumoral CD45+ from MCA205 (day 19) from WT and Trem2KO mice. B. Heatmap showing enrichment of transcription factors binding sites in the regulatory regions of 12 metacell marker genes. Only TFs with significant Normalized Enrichment Score (NES>3.5) and average expression above 0.1 UMI in metacell are shown. C. MCA205 tumor images from WT (upper tumor image) and Trem2 KO (lower tumor image).

### DESCRIPTION OF SPECIFIC EMBODIMENTS OF THE INVENTION

The present invention, in some embodiments thereof, relates to a method of treating cancer by reducing the immune suppressor activity of myeloid cells and, more particularly, but not exclusively, to solid cancers.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors analyzed suppressive metabolic circuits within the tumor microenvironment using the direct targeting of Arg1⁺ myeloid cells. They identified two distinct populations of Arg1⁺ Trem2⁺ cells in the tumor, a tumor associated macrophage population and a unique population of Mreg, characterized by defined surface markers (e.g. Gpnmb), and signaling, including hypoxia. They demonstrated the suppressive activity of the Arg1⁺ TAM and Mreg populations over CD8 T cells. The present findings identified Trem2 as a marker and potential regulator of suppressive myeloid cells. Genetic ablation of Trem2 in mice, led to dramatic decrease in the Mreg population with increase in immune reactivity towards the tumor, including decrease in dysfunctional CD8⁺ T cells and increase in NK and cytotoxic T cells. The results suggest that specific targeting of the Mreg population will be more beneficial than targeting the tumor associated macrophage population for the treatment of cancer.

Thus, according to a first aspect of the present invention there is provided a method of reducing the immune suppressor activity of myeloid cells, the method comprising contacting myeloid cells with an effective amount of an agent which specifically reduces the amount and/or activity of myeloid cells expressing both Triggering Receptor Expressed On Myeloid Cells 2 (Trem2) and Transmembrane glycoprotein NMB (Gpnmb), thereby reducing the immune suppressor activity of myeloid cells.

The term “myeloid cells” as used herein refers to cells which arise from the common myeloid progenitor (CMP). In one embodiment, myeloid cells are ones which, arise from the lineage of the myeloblast and their daughter types (e.g. basophils, neutrophils, eosinophils, monocytes and macrophages). One subgroup of myeloid cells are immune suppressor myeloid cells.

TREM-2 is an immunoglobulin-like receptor primarily expressed on myeloid lineage cells, including without limitation, macrophages, dendritic cells, osteoclasts, microglia, monocytes, Langerhans cells of skin, and Kupffer cells. In some embodiments, TREM-2 forms a receptor-signaling complex with DAP12. In some embodiments, TREM-2 phosphorylates and signals through DAP12 (an ITAM domain adaptor protein). In some embodiments TREM-2 signaling results in the downstream activation of PI3K. In some embodiments TREM-2 signaling results in the downstream phosphorylation of spleen tyrosine kinase (stk).

TREM-2 proteins of the present disclosure include, without limitation, a mammalian TREM-2 protein including but not limited to human TREM-2 protein (Uniprot Accession No. Q9NZC2), mouse TREM-2 protein (Uniprot Accession No. Q99NH8), rat TREM-2 protein (Uniprot Accession No. D3ZZ89), Rhesus monkey TREM-2 protein (Uniprot Accession No. F6QVF2), bovine TREM-2 protein (Uniprot Accession No. Q05B59), equine TREM-2 protein (Uniprot Accession No. F7D6L0), pig TREM-2 protein (Uniprot Accession No. H2EZZ3), and dog TREM-2 protein (Uniprot Accession No. E2RP46).

An exemplary human TREM-2 amino acid sequence is set forth below as SEQ ID NO: 49.

In some embodiments, the human TREM-2 is a preprotein that includes a signal peptide. In some embodiments, the human TREM-2 is a mature protein. In some embodiments, the mature TREM-2 protein does not include a signal peptide. In some embodiments, the mature TREM-2 protein is expressed on a cell. In some embodiments, TREM-2 contains a signal peptide located at amino acid residues 1-18 of human TREM-2 (SEQ ID NO: 49); an extracellular immunoglobulin-like variable-type (IgV) domain located at amino acid residues 29-112 of human TREM-2 (SEQ ID NO: 49); additional extracellular sequences located at amino acid residues 113-174 of human TREM-2 (SEQ ID NO: 49); a transmembrane domain located at amino acid residues 175-195 of human TREM-2 (SEQ ID NO: 49); and an intracellular domain located at amino acid residues 196-230 of human TREM-2 (SEQ ID NO: 49).

Transmembrane glycoprotein NMB (GPNMB) is a type IA cell-surface glycoprotein. An exemplary GPNMB has an amino acid sequence as set forth in SEQ ID NO: 50. In one embodiment, the term GPNMB refers to an analog, derivative or a fragment thereof, or a fusion protein comprising GPNMB, an analog, derivative or a fragment thereof. In certain embodiments, the term “GPNMB” refers to the mature, processed form of GPNMB. In other embodiments, the term “GPNMB” refers to the extracellular domain of GPNMB.

As mentioned, an agent is contacted with myeloid cells of the subject in order to reduce the amount and/or activity of a specific subpopulation of said myeloid cells—those expressing both Trem2 and Gpnmb.

In one embodiment, the contacting is carried out in vivo.

In another embodiment, the contacting is carried out ex vivo—i.e. myeloid cells are removed from a subject and subsequently contacted with the agent.

Myeloid cells are typically removed from subjects by bone marrow biopsy.

The agent of this aspect of the present invention specifically reduces the amount and/or activity of myeloid cells expressing both Triggering Receptor Expressed On Myeloid Cells 2 (Trem2) and Transmembrane glycoprotein NMB (Gpnmb).

In one embodiment, the agent reduces the amount of cells expressing both markers at least 2 fold compared to cells expressing only one of the markers (i.e. only TREM2 and not Gpnmb or vice versa). In another embodiment, the agent reduces the amount of cells expressing both markers at least 5 fold compared to cells expressing only one of the markers (i.e. only TREM2 and not Gpnmb or vice versa). In another embodiment, the agent reduces the amount of cells expressing both markers at least 10 fold compared to cells expressing only one of the markers (i.e. only TREM2 and not Gpnmb or vice versa).

In one embodiment, the agent reduces the activity of cells expressing both markers at least 2 fold compared to cells expressing only one of the markers (i.e. only TREM2 and not Gpnmb or vice versa). In another embodiment, the agent reduces the activity of cells expressing both markers at least 5 fold compared to cells expressing only one of the markers (i.e. only TREM2 and not Gpnmb or vice versa). In another embodiment, the agent reduces the activity of cells expressing both markers at least 10 fold compared to cells expressing only one of the markers (i.e. only TREM2 and not Gpnmb or vice versa).

The agent of this aspect of the present invention described herein may bind to both TREM-2 and to Gpnmb with a KD of <1×10⁻⁷ M. In yet another embodiment, the antigen binding proteins of the invention bind to both human TREM-2 and to human Gpnmb with a KD of <5×10⁻⁸ M. In another embodiment, the antigen binding proteins of the invention bind to both human TREM-2 and to human Gpnmb with a KD of <1×10⁻⁸ M. In certain embodiments, the antigen binding proteins of the invention bind to both human TREM-2 and to human Gpnmb with a KD of <5×10⁻⁹ M. In other embodiments, the antigen binding proteins of the invention bind to both human TREM-2 and to human Gpnmb with a KD of <1×10⁻⁹ M. In one particular embodiment, the antigen binding proteins of the invention bind to both human TREM-2 and to human Gpnmb with a KD of <5×10⁻¹⁰ M. In another particular embodiment, the antigen binding proteins of the invention bind to both human TREM-2 and to human Gpnmb with a KD of <1×10⁻¹⁰ M. In another particular embodiment, the antigen binding proteins of the invention bind to both human TREM-2 and to human Gpnmb with a KD of <1×10⁻¹¹ M. In another particular embodiment, the antigen binding proteins of the invention bind to both human TREM-2 and to human Gpnmb with a KD of <1×10⁻¹² M. In another particular embodiment, the antigen binding proteins of the invention bind to both human TREM-2 and to human Gpnmb with a KD of <1×10⁻¹³ M. In another particular embodiment, the antigen binding proteins of the invention bind to both human TREM-2 and to human Gpnmb with a KD of <1×10⁻¹⁴ M. In another particular embodiment, the antigen binding proteins of the invention bind to both human TREM-2 and to human Gpnmb with a KD of <1×10⁻¹⁵ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (ka) and the dissociation rate constant (kd) can be measured. The equilibrium dissociation constant (KD in M) can then be calculated from the ratio of the kinetic rate constants (kd/ka). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (KD in M) and the association rate constant (ka in M′V¹) can be measured. The dissociation rate constant (kd) can be calculated from these values (KD X ka). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (ka and kd) and affinity (KD) constants can be calculated in real-time using the bio-layer interferometry method. In some embodiments, the antigen binding proteins described herein exhibit desirable characteristics such as binding avidity as measured by kd (dissociation rate constant) for human TREM-2 and for human Gpnmb of about 10⁻², 10⁻³, 10⁻⁴, 10⁻⁵, 10⁻⁶ or lower (lower values indicating higher binding avidity), and/or binding affinity as measured by KD (equilibrium dissociation constant) for human TREM-2 and for human Gpnmb of about 10⁻⁸, 10⁻⁹, 10⁻¹⁰, 10⁻¹¹ M or lower (lower values indicating higher binding affinity). In certain embodiments, the antigen binding proteins of the invention specifically bind to human TREM-2 and for human Gpnmb with a KD from about 1 pM to about 100 nM as measured by bio-layer interferometry at 25° C. For instance, in some embodiments, the antigen binding proteins of the invention specifically bind to human TREM-2 and for human Gpnmb with a KD less than 100 nM as measured by bio-layer interferometry at 25° C. In other embodiments, the antigen binding proteins of the invention specifically bind to human TREM-2 and for human Gpnmb with a KD less than 50 nM as measured by bio-layer interferometry at 25° C. In yet other embodiments, the antigen binding proteins of the invention specifically bind to human TREM-2 and for human Gpnmb with a KD less than 25 nM as measured by bio-layer interferometry at 25° C. In one particular embodiment, the antigen binding proteins of the invention specifically bind to human TREM-2 and for human Gpnmb with a KD less than 10 nM as measured by bio-layer interferometry at 25° C. In another particular embodiment, the antigen binding proteins of the invention specifically bind to human TREM-2 and for human Gpnmb with a KD less than 5 nM as measured by bio-layer interferometry at 25° C. In another particular embodiment, the antigen binding proteins of the invention specifically bind to human TREM-2 and for human Gpnmb with a KD less than 1 nM as measured by bio-layer interferometry at 25° C.

According to one embodiment, the agent of this aspect of the present invention is a bispecific antibody recognizing two different antigens, TREM-2 and Gpnmb, a multivarient antibody or a chimeric antibody.

“Bi-specific antibody” of the present invention has two different antigen binding sites, such that the antibody specifically binds to two different antigens. Such antibodies may be generated by combining parts of two separate antibodies or antibody fragments that recognize two different antigenic groups or modifying a single antibody molecule to comprise two specificities (as discussed in detail hereinabove).

According to one embodiment, the bi-specific antibody is a hybrid antibody having two different heavy/light chain pairs and two different binding sites.

According to one embodiment, the bi-specific antibody comprises an antigen recognition domain in a structural loop region of the antibody (e.g. CH3 region of the heavy chain).

Accordingly, the bi-specific antibody may comprise an antibody fragment comprising a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Antibodies having higher valencies (i.e., the ability to bind to more than two antigens) can also be prepared; they are referred to as multispecific antibodies.

Since the method described herein is used to reduce the immune suppressor activity of myeloid cells, the present inventors conceive that the method may be used to treat cancer.

Thus, according to another aspect of the present invention there is provided a method of treating cancer in a subject in need thereof, the method comprising administering to the subject an effective amount of an agent which specifically downregulates the amount and/or the activity of myeloid cells of the subject expressing both Triggering Receptor Expressed On Myeloid Cells 2 (Trem2) and Transmembrane glycoprotein NMB (Gpnmb), thereby treating the cancer.

As used herein “subject” refers to a mammal, e.g., human, diagnosed with cancer.

Agents capable of specifically decreasing the amount and/or the activity of myeloid cells of the subject which express Trem2 and Gpnmb are described herein above.

The terms “cancer” and “cancerous” refer to or describe the physiological condition in mammals that is typically characterized by unregulated malignant cell growth.

Examples of cancers that can be analyzed and treated according to some embodiments of the invention, include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to a specific embodiment, the cancer is melanoma.

According to a specific embodiment, the cancer is a solid tumor (lung cancer, liver cancer, ovarian cancer, gastric cancer and breast cancer).

According to a specific embodiment, the cancer is a primary tumor.

According to a specific embodiment, the cancer is metastatic.

According to a specific embodiment, the cancer is a secondary tumor.

According to a specific embodiment, the lung cancer is non-small cell lung cancer.

According to a specific embodiment, the lung cancer is small cell lung cancer.

According to a specific embodiment, the liver cancer is Hepatocellular carcinoma.

According to another aspect of the present invention there is provided a method of treating cancer in a subject in need thereof, the method comprising administering to the subject a therapeutically effective amount of

(i) a first agent which down-regulates the amount and/or activity of Trem2; and

(ii) a second agent which specifically down-regulates the amount and/or activity of Gpnmb, thereby treating the cancer.

According to one embodiment, the first agent binds specifically to TREM-2 which is expressed on myeloid cells. According to another embodiment, the second agent binds specifically to Gpnmb. The phrase “specifically bind(s)” or “bind(s) specifically” when referring to a binding molecule refers to a binding molecule which has intermediate or high binding affinity, exclusively or predominately, to a target molecule, such as to TREM-2 or Gpnmb. The phrase “specifically binds to” refers to a binding reaction which is determinative of the presence of a target protein (such as TREM-2 or Gpnmb) in the presence of a heterogeneous population of proteins and other biologics. Thus, under designated assay conditions, the specified binding molecules bind preferentially to a particular target protein (e.g. TREM-2 or Gpnmb) and do not bind in a significant amount to other components present in a test sample. Specific binding to a target protein under such conditions may require a binding molecule that is selected for its specificity for a particular target protein. A variety of assay formats may be used to select binding molecules that are specifically reactive with a particular target protein. For example, solid-phase ELISA immunoassays, immunoprecipitation, Biacore and Western blot may be used to identify binding molecules that specifically bind to TREM-2 or Gpnmb. Typically, a specific or selective reaction will be at least twice background signal or noise and more typically more than 10 times background. Given that the binding molecule is an antibody, the phrase “specifically binds to” refers to a binding reaction that is determinative of the presence of the antigen (such as TREM-2 or Gpnmb) in a heterogeneous population of proteins and other biologics. Typically, an agent that specifically binds to an antigen binds the antigen with a dissociation constant (K_{D}) of at least about 1×10⁻⁶ to 1×10⁻⁷, or about 1×10⁻⁸ to 1×10⁻⁹ M, or about 1×10⁻¹° to 1×10⁻¹¹ or higher; and/or binds to the predetermined antigen (e.g. of TREM-2 or Gpnmb) with an affinity that is at least two-fold, five-fold, ten-fold, twenty-fold greater than its affinity for binding to a non-specific antigen (e.g., BSA, casein) other than the predetermined antigen or a closely-related antigen.

According to a particular embodiment, the agent which decreases the amount and/or activity of TREM-2 is an inhibitor antibody, also referred to herein as an antagonist antibody. The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

In a particular embodiment, the antibody is a monoclonal antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference and the Examples section which follows).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Examples of TREM-2 inhibitor antibodies are disclosed in WO 2017/058866 and US Application NO. 20190010230, the contents of which are disclosed herein by reference.

Other exemplary TREM-2 inhibitor antibodies are disclosed in US Patent Application No. 20200017584 and US Patent Application No. 20190336615.

An example of a TREM-2 inhibitor antibody is one which has a CDR-H1 of SEQ ID NO:51, a CDR-H2 of SEQ ID NO: 52, a CDR-H3 of SEQ ID NO: 53, a CDR-L1 of SEQ ID NO: 54, a CDR-L2 of SEQ ID NO:55, and a CDR-L1 of SEQ ID NO:56.

Additional TREM-2 antibodies contemplated by the present inventors include those commercially available from R&D including BAF1828 (human), MAB17291 (human and mouse), AF1828 (human), and AF1729 (mouse).

According to a particular embodiment, the agent which decreases the amount and/or activity of Gpnmb is an inhibitor antibody, also referred to herein as an antagonist antibody.

Examples of Gpnmb inhibitory antibodies are disclosed in US Application No. 20180043014, the contents of which are disclosed herein by reference.

Exemplary human anti-GPNMB antibodies include Mab1.10.2, Mab1.15.1, Mab1.2.2, Mab1.7.1, Mab2.10.2, Mab2.15.1, Mab2.16.1, Mab2.17.1. These antibodies have amino acid sequences and nucleic acid sequences encoding them identified in this application as shown in Tables 1 and 2.

TABLE 1
Antibody Nucleotide (DNA) and Amino Acid (AA) Sequences

| Gene Segment | 1.10.2 | 1.15.1 | 1.2.2 | 1.7.1 |
|---|---|---|---|---|
| H CDR1 | SEQ ID | SEQ ID | SEQ ID | SEQ ID |
| | NO: 1 | NO: 7 | NO: 13 | NO: 19 |
| H CDR2 | SEQ ID | SEQ ID | SEQ ID | SEQ ID |
| | NO: 2 | NO: 8 | NO: 14 | NO: 20 |
| H CDR3 | SEQ ID | SEQ ID | SEQ ID | SEQ ID |
| | NO: 3 | NO: 9 | NO: 15 | NO: 21 |
| L CDR1 | SEQ ID | SEQ ID | SEQ ID | SEQ ID |
| | NO: 4 | NO: 10 | NO: 16 | NO: 22 |
| L CDR2 | SEQ ID | SEQ ID | SEQ ID | SEQ ID |
| | NO: 5 | NO: 11 | NO: 17 | NO: 23 |
| L CDR3 | SEQ ID | SEQ ID | SEQ ID | SEQ ID |
| | NO: 6 | NO: 12 | NO: 18 | NO: 24 |

TABLE 2
Antibody Nucleotide (DNA) and Amino Acid (AA) Sequences

| Gene Segment | 2.10.2 | 2.15.1 | 2.16.1 | 2.17.1 |
|---|---|---|---|---|
| H CDR1 | SEQ ID | SEQ ID | SEQ ID | SEQ ID |
| | NO: 25 | NO: 31 | NO: 37 | NO: 43 |
| H CDR2 | SEQ ID | SEQ ID | SEQ ID | SEQ ID |
| | NO: 26 | NO: 32 | NO: 38 | NO: 44 |
| H CDR3 | SEQ ID | SEQ ID | SEQ ID | SEQ ID |
| | NO: 27 | NO: 33 | NO: 39 | NO: 45 |
| L CDR1 | SEQ ID | SEQ ID | SEQ ID | SEQ ID |
| | NO: 28 | NO: 34 | NO: 40 | NO: 46 |
| L CDR2 | SEQ ID | SEQ ID | SEQ ID | SEQ ID |
| | NO: 29 | NO: 35 | NO: 41 | NO: 47 |
| L CDR3 | SEQ ID | SEQ ID | SEQ ID | SEQ ID |
| | NO: 30 | NO: 36 | NO: 42 | NO: 48 |

The antibodies described herein (both bispecific or monospecific) may be attached to cytotoxic agents.

As used herein, the term “cytotoxic agent” refers to refers to a substance that inhibits or prevents the function of cells and/or causes destruction of cells. The term is intended to include radioactive isotopes (e.g. ²¹¹At, ¹³¹I, ¹²⁵I, ³²P, ³⁵S and radioactive isotopes of Lu, including ¹⁷⁷Lu, ⁸⁶Y, ⁹⁰Y, ¹¹¹In, ¹⁷⁷Lu, ²²⁵Ac, ²¹²Bi, ²¹³Bi, ⁶⁶Ga, ⁶⁷Ga, ⁶⁸Ga, ⁶⁴cu, ⁶⁷Cu, ⁷¹As, ⁷²As, ⁷⁶As, ⁷⁷As, ⁶⁵Zn, ⁴⁸V, ²⁰³Pb, ²⁰⁹Pb, ²¹²Pb, ¹⁶⁶Ho, ¹⁴⁹Pm, ¹⁵³Sm, ²⁰¹Tl, ¹⁸⁸Re, ¹⁸⁶Re and ⁹⁹mTc), anticancer agents as otherwise described herein, including chemotherapeutic (anticancer drugs e.g. methotrexate, adriamicin, vinca alkaloids (vincristine, vinblastine, etoposide), taxol, doxoruicin, cisplatin, 5-fluorouridine, melphalan, ethidium bromide, mitomycin C, chlorambucil, daunorubicin and other intercalating agents, enzymes and fragments thereof such as nucleolytic enzymes, antibiotics, therapeutic RNA molecules (e.g., siRNA, antisense oligonucleotides, microRNA, ribozymes, RNA decoys, aptamers), DNAzymes, antibodies, proteins and polynucleotides encoding same, and toxins such as small molecule toxins or enzymatically active toxins of bacterial, fungal, plant or animal origin, such as pokeweed antiviral protein (PAP), ricin toxin A, abrin, gelonin, saporin, cholera toxin A, diphtheria toxin, Pseudomonas exotoxin, and alpha-sarcin, including fragments and/or variants thereof.

It will be appreciated that the agent of the present invention (e.g., the antibody) can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent of the present invention (e.g., the antibody) which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, neurosurgical strategies (e.g., intracerebral injection, intrastriatal infusion or intracerebroventricular infusion, intra spinal cord, epidural), transmucosal, intestinal or parenteral delivery, including intramuscular, subcutaneous and intramedullary injections as well as intrathecal, direct intraventricular, intracardiac, intravenous, intraperitoneal, intranasal, or intraocular injections.

Alternately, one may administer the pharmaceutical composition in a local rather than a systemic manner, for example, via injection of the pharmaceutical composition directly into a tissue region of a patient (e.g. adipose tissue).

According to a preferred embodiment, the agents are not administered into the brain of the subject.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose (e.g. reduction of number or size of adipocytes, or decrease in the amount of visceral fat).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide tissue levels of the active ingredient that are sufficient to decrease the number or size of adipocytes or decrease visceral fat (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The present inventors contemplate administering to the subject (in combination with the above described agents that target the TREM-2/Gpnmb expressing cells) additional chemotherapeutic agents. Such agents may work synergistically with the above described agents for the treatment of cancer.

Treatment can be combined with any anti-cancer treatment known in the art, including, but not limited to, chemotherapeutic agents, radiotherapeutic agents, hormonal therapy, immune modulators, engineered immune cell therapy (e.g., CAR-T) and other treatment regimens (e.g., surgery, cell transplantation e.g. hematopoietic stem cell transplantation) which are well known in the art.

The chemotherapeutic agent of the present invention can be, but not limited to, cytarabine (cytosine arabinoside, Ara-C, Cytosar-U), asprin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2 difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including Nmethylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

In some embodiments the chemotherapeutic agent of the present invention is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, Cytosar-U), melphalan, busulfan (Myleran®), thiotepa, bleomycin, platinum (cisplatin), cyclophosphamide, Cytoxan®)., daunorubicin, doxorubicin, idarubicin, mitoxantrone, 5-azacytidine, cladribine, fludarabine, hydroxyurea, 6-mercaptopurine, methotrexate, 6-thioguanine, or any combination thereof.

According to a specific embodiment, the treatment is combined with an immune checkpoint inhibitor, such as described below.

As used herein “immune checkpoint inhibition” refers to cancer immunotherapy. The therapy targets immune checkpoints, key regulators of the immune system that stimulate or inhibit its actions, which tumors can use to protect themselves from attacks by the immune system. Checkpoint therapy can block inhibitory checkpoints, activate stimulatory functions, thereby restoring immune system function. Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274).

Examples of immune checkpoint inhibitors include, but are not limited to, of cytotoxic T-lymphocyte antigen 4 (CTLA4), programmed death 1 (PD-1) or its ligands, lymphocyte activation gene-3 (LAGS), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD244 (2B4), CD160, GARP, OX40, CD137 (4-1BB), CD25, VISTA, BTLA, TNFR25, CD57, CCR2, CCRS, CCR6, CD39, CD73, CD4, CD18, CD49b, CD1d, CDS, CD21, TIMI, CD19, CD20, CD23, CD24, CD38, CD93, IgM, B220 (CD45R), CD317, CD11b, Ly6G, ICAM-1, FAP, PDGFR, Podoplanin, and TIGIT.

Examples of clinically approved immune checkpoint inhibitors include, but are not limited to, Ipilimumab, (anti CTLA-4), Nivolimumab (anti PD-1) and Pembrolizumab (anti PD 1).

According to another embodiment, the treatment is combined with a Brutons tyrosine kinase (Btk) inhibitor (e.g. ibrutinib, acalabrutinib or Spebrutinib).

The present inventors also contemplate selecting a treatment type based on the presence of myeloid cells which express both Trem2 and Gpnmb.

Thus, according to still another aspect of the present invention there is provided a method of treating cancer in a subject comprising:

(a) analyzing in a sample of the subject for the presence of myeloid cells which express both Trem2 and Gpnmb; and

(b) when there is an amount of said myeloid cells above a predetermined amount, treating the subject with a therapeutically effective amount of an agent that targets Trem2 and/or Gpnmb; or when there is an amount of said cells below a predetermined amount treating the subject with a therapeutically effective amount of a chemotherapeutic agent other than said agent that targets Trem2 and/or Gpnmb.

Methods of determining gene expression profiles can be performed at the RNA or protein level.

Below is a more detailed description of methods that can be used to analyze expression of a plurality of genes on the single cell level.

Methods of Analyzing and/or Quantifying RNA

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo GJ, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Single Cell Transcriptome Analysis

This method relies on sequencing the transcriptome of a single cell. In one embodiment a high-throughput method is used, where the RNAs from different cells are tagged individually, allowing a single library to be created while retaining the cell identity of each read. The method can be carried out a number of ways—see for example US Patent Application No. 20100203597 and US Patent Application No. 20180100201, the contents of which are incorporated herein by reference.

One particular method for carrying out single cell transcriptome analysis is summarized below.

Cells are typically aliquoted into wells such that only one cell is present per well. Cells are treated with an agent that disrupts the cell and nuclear membrane making the RNA of the cell accessible to sequencing reactions.

According to one embodiment, the RNA is amplified using the following in vitro transcription amplification protocol:

(Step 1) contacting the RNA of a single cell with an oligonucleotide comprising a polydT sequence at its terminal 3′ end, a T7 RNA polymerase promoter sequence at its terminal 5′ end and a barcode sequence positioned between the polydT sequence and the RNA polymerase promoter sequence under conditions that allow synthesis of a single stranded DNA molecule from the RNA, wherein the barcode sequence comprises a cell barcode and a molecular identifier;

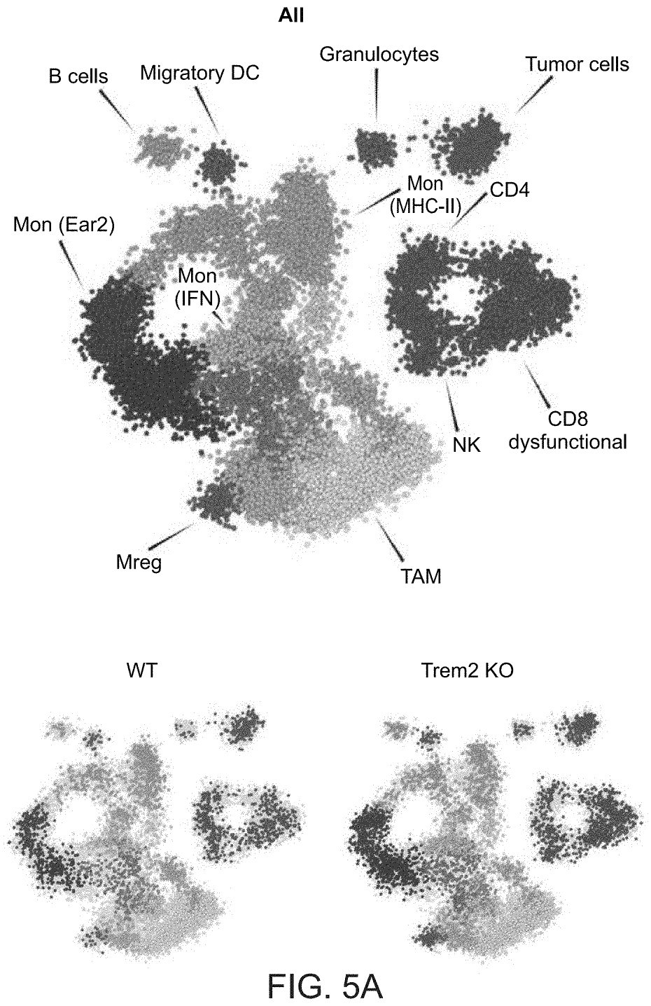

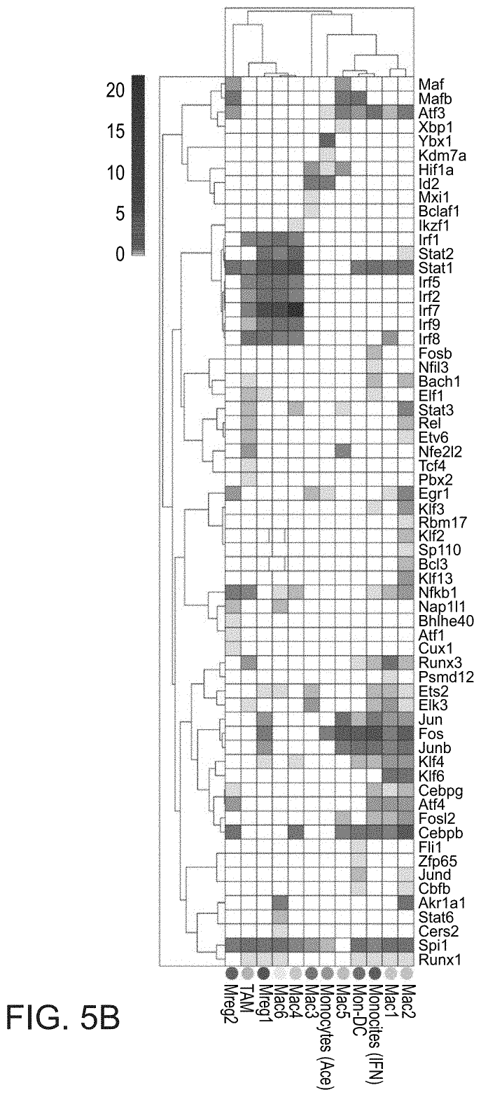

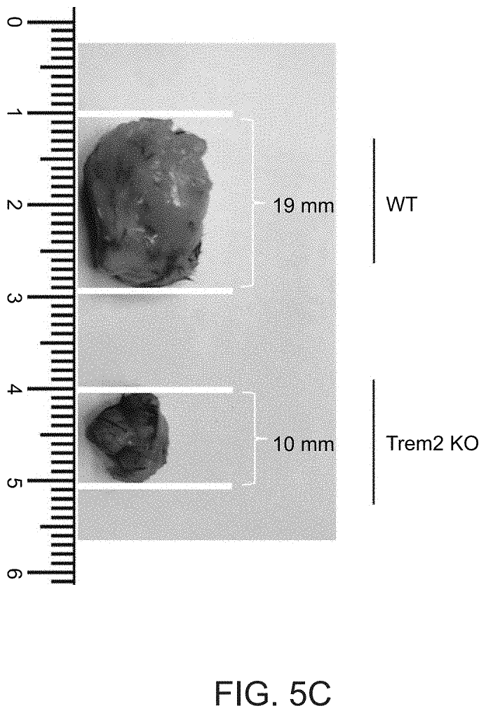

The polydT oligonucleotide of this embodiment may optionally comprise an adapter sequence required for sequencing—see for example FIGS. 5A-C.

RNA polymerase promoter sequences are known in the art and include for example T7 RNA polymerase promoter sequence—e.g. SCGATTGAGGCCGGTAATACGACTCACTATAGGGGC (SEQ ID NO: 57).

Preferably the polydT sequence comprises at least 5 nucleotides. According to another embodiment the polydT sequence is between about 5 to 50 nucleotides, more preferably between about 5-25 nucleotides, and even more preferably between about 12 to 14 nucleotides.

The barcode sequence is useful during multiplex reactions when a number of samples are pooled in a single reaction. The barcode sequence may be used to identify a particular molecule, sample or library. The barcode sequence is attached 5′ end of polydT sequence and 3′ of the T7 RNA polymerase sequence. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides.

In one embodiment, the barcode sequence is used to identify a cell type, or a cell source (e.g. a patient).

Molecular identifiers are useful to correct for amplification bias, which reduces quantitative accuracy of the method. The molecular identifier comprises between 4-20 bases. The molecular identifier is of a length such that each RNA molecule of the sample is catalogued (labeled) with a molecular identifier having a unique sequence.

Following annealing of a primer (e.g. polydT primer) to the RNA sample, an RNA-DNA hybrid may be synthesized by reverse transcription using an RNA-dependent DNA polymerase. Suitable RNA-dependent DNA polymerases for use in the methods and compositions of the invention include reverse transcriptases (RTs). RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules. However, in some instances, it is preferable to employ a RT which lacks or has substantially reduced RNase H activity.

RTs devoid of RNase H activity are known in the art, including those comprising a mutation of the wild type reverse transcriptase where the mutation eliminates the RNase H activity. Examples of RTs having reduced RNase H activity are described in US20100203597. In these cases, the addition of an RNase H from other sources, such as that isolated from E. coli, can be employed for the formation of the single stranded cDNA. Combinations of RTs are also contemplated, including combinations of different non-mutant RTs, combinations of different mutant RTs, and combinations of one or more non-mutant RT with one or more mutant RT.

Examples of suitable enzymes include, but are not limited to AffinityScript from Agilent or Superscript III from Invitrogen. Preferably the reverse transcriptase is devoid of terminal Deoxynucleotidyl Transferase (TdT) activity.

Additional components required in a reverse transcription reaction include dNTPS (dATP, dCTP, dGTP and dTTP) and optionally a reducing agent such as Dithiothreitol (DTT) and MnC12.

The polydT oligonucleotide may be attached to a solid support (e.g. beads) so that the cDNA which is synthesized may be purified.

Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

(Step 2): Once cDNA is generated, the cDNA may be pooled from cDNA generated from other single cells (using the same method as described herein above).

The sample may optionally be treated with an enzyme to remove excess primers, such as exonuclease I. Other options of purifying the single stranded DNA are also contemplated including for example the use of paramagnetic microparticles. This may be carried out following or prior to sample pooling.

(Step 3): Second Strand Synthesis.

Second strand synthesis of cDNA may be effected by incubating the sample in the presence of nucleotide triphosphates and a DNA polymerase. Commercial kits are available for this step which include additional enzymes such as RNAse H (to remove the RNA strand) and buffers. This reaction may optionally be performed in the presence of a DNA ligase. Following second strand synthesis, the product may be purified using methods known in the art including for example the use of paramagnetic microparticles.

(Step 4): Following synthesis of the second strand of the cDNA, RNA may be synthesized by incubating with a corresponding RNA polymerase. Commercially available kits may be used such as the T7 High Yield RNA polymerase IVT kit (New England Biolabs).

(Step 5): Prior to fragmentation of the amplified RNA, the DNA may be removed using a DNAse enzyme. The RNA may be purified as well prior to fragmentation. Fragmentation of the RNA may be carried out as known in the art. Fragmentation kits are commercially available such as the Ambion fragmentation kit.

(Step 6): The amplified and fragmented RNA is now labeled on its 3′ end. For this a ligase reaction is performed which essentially ligates single stranded DNA (ssDNA) to the RNA. Other methods of labeling the amplified and fragmented RNA are described in US Application No. 20170137806, the contents of which are incorporated herein by reference. The single stranded DNA has a free phosphate at its 5′end and optionally a blocking moiety at its 3′end in order to prevent head to tail ligation. Examples of blocking moieties include C3 spacer or a biotin moiety. Typically, the ssDNA is between 10-50 nucleotides in length and more preferably between 15 and 25 nucleotides.

(Step 7): Reverse transcription is then performed using a primer that is complementary to the primer used in the preceding step. The library may then be completed and amplified through a nested PCR reaction as illustrated in FIGS. 5A-C.

(Step 8): Amplification

Once the adapter polynucleotide of the present invention is ligated to the single stranded DNA (i.e. further to extension of the single stranded DNA), amplification reactions may be performed.

(Step 9): Sequencing

Methods for sequence determination are generally known to the person skilled in the art. Preferred sequencing methods are next generation sequencing methods or parallel high throughput sequencing methods e.g. Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

It will be appreciated that methods which rely on microfluidics can also be used to carry out single cell transcriptome analysis.

Thus, a combination of molecular barcoding and emulsion-based microfluidics to isolate, lyse, barcode, and prepare nucleic acids from individual cells in high-throughput may be used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode about 10,000-100,000 cells. In this regard there can be a single-cell sequencing library which may comprise: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells: and preparing and sequencing a single composite RNA-Seq library, as described herein above. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2, all the contents and disclosure of each of which are herein incorporated by reference in their entirety.

Methods of Detecting Expression and/or Activity of Proteins

Expression and/or activity level of proteins expressed in the cells of the cultures of some embodiments of the invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to a specific embodiment, the gene expression is determined by transcriptome analysis.

According to a specific embodiment, the gene expression is determined by a single cell transcriptome analysis as described above.

Thus, once a particular level of cells is observed e.g., more than 5%, more than 10% of myeloid cells of a sample derived from the, the subject can be considered as a candidate for a therapy that targets these cells. If an insufficient number of myeloid cells of this signature is observed, the subject is not considered as a candidate for this therapy.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.
Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

INs-Seq Fixation and Intracellular Staining Protocol:

1—Cell surface staining: Cells or tissue from in vitro and in vivo experiments respectively were dissociated into single cell suspension and washed with 10 ml cold PBS. Cells were stained in ice cold washing buffer (−/− Dulbecco's Phosphate Buffered Saline (Biological industries), 0.5% BSA (MP-Biomedicals), 2 mM EDTA (Merck)) with fluorophores conjugated antibodies (final concentration of 5 μg/mL) on ice for 30 minutes in the dark.

2—INs-seq fixation: Surface-stained cells were washed in 10 ml washing buffer and centrifuged at 400 g for 5 minutes. Cell pellet (1×10^{\hat{ }6}-5×10^{\hat{ }6} cells) was resuspended in 1 volume (100 μl) of cold PBS (0.4 U/μL RNasin® Plus RNase Inhibitor (Promega)). Cell suspension was fixed with 9 volumes (900 μl) of cold 100% methanol (Bio-Lab) (pre-chilled to −20° C.) for 10 minutes on ice in the dark. To avoid cell clumping, methanol was added in drops, while gently vortexing the cell suspension.

Fixed cells were pelleted at 900 g for 3 minutes right after fixation. Methanol-PBS solution was completely discarded. Cell pellet was washed (not resuspended) twice with ice-cold PBS (0.4 U/μL RNasin® Plus RNase Inhibitor) without breaking the pellet, for complete removal of methanol leftovers. Cell pellet was resuspended in 100 μL of enzyme blocking buffer containing ammonium sulfate solution (Thermo Fisher), 0.05M EDTA (Sigma), 0.8 U/μL RNasin® Plus RNase Inhibitor, pH of 5.2) and kept on ice for 10 minutes in the dark.

3—Intracellular staining: To wash enzyme blocking buffer solution, 1 ml washing buffer (0.4 U/μL RNasin® Plus RNase inhibitor) was added, cells then were pelleted at 900 g for 3 minutes. To completely remove enzyme blocking buffer, cell pellet was washed twice with ice-cold washing buffer (0.4 U/μL RNasin® Plus RNase inhibitor) without re-suspension. Cell pellet was then incubated in the dark for 20 minutes with 100 μl intracellular staining buffer (−/− Dulbecco's Phosphate Buffered Saline (Biological industries), 0.5% BSA (MP biochemical), 2M EDTA (Sigma) with the desired intracellular antibody). At the end of the incubation, 1 ml washing buffer (0.4 U/μL RNasin® Plus RNase inhibitor) was added on top of the 100 μl intracellular staining buffer, cells were pelleted at 900 g for 3 minutes. Cell pellet was resuspended in 1 ml preservation buffer, filtered with 70 um nylon mesh and kept on ice until cell sorting.

Fixation and Intracellular Staining Methods

1—Cell surface staining: Cells or tissue from in vitro and in vivo experiments respectively were dissociated into single cell suspension and washed with 10 ml cold PBS. Cells were stained in ice cold washing buffer (−/− Dulbecco's Phosphate Buffered Saline (Biological industries), 0.5% BSA (MP-Biomedicals), 2 mM EDTA (Merck)) with fluorophores conjugated antibodies (final concentration of 5 m/mL) on ice for 30 minutes in the dark.

2—Fixation methods: Methanol based cell fixation protocol: adopted from (Alles et al. 2017). Surface-stained cells were washed in 10 ml washing buffer and centrifuged at 400 g for 5 minutes. Cells were handled in regular microcentrifuge tubes to minimize cell loss and kept cold at all times. Cells were resuspended in 100 μl of ice-cold PBS. To avoid cell clumping, 8 volumes (800 μl) of methanol (pre-chilled to −20° C.) were added dropwise, while gently mixing or vortexing the cell suspension (final concentration: 90% methanol in PBS). The methanol-fixed cells were kept on ice for a minimum of 15 min. For rehydration, cells were pelleted at 900 g for 4 min, rehydrated in PBS (0.01% BSA, 1 U/μl RNasin® Plus RNase inhibitor) pelleted, centrifuged, resuspended again in PBS (0.01% BSA, 1 U/μl RNAse inhibitor) and filtered with 70 um nylon mesh and kept on ice until cell sorting.

PFA based—Surface-strained cells were washed in 10 ml washing buffer and centrifuged at 400 g for 5 minutes. True-Nuclear Transcription Factor Buffer Set commercial kit was done according to the published protocol. Cells were filtered with 70 um nylon mesh and kept on ice until cell sorting.

DSP based cell fixation protocol: was adopted from (Gerlach et al. 2019). Surface-srained cells were washed in 10 ml washing buffer and centrifuged at 400 g for 5 minutes. Cells were fixed using a combination of 2.5 mM DSP (Thermo Scientific) and 2.5 mM SPDP (Thermo Scientific) in DMSO for 45 minutes in 200 mM Sodium Phosphate Buffered Saline pH 8.4 (1M stock solution of Sodium-Phosphate buffer includes 1M NaH2PO4 (Sodium phosphate monobasic (Sigma)) and 1M Na2HPO4 (Sodium phosphate dibasic (Sigma)) solutions), and 150 mM NaCl (Sigma). After fixative quenching with 100 mM Tris-HCl pH 7.5, 150 mM NaCl the cells were blocked and permeabilized using 0.5X Protein Free Blocking Buffer (PFBB, Thermo scientific) in PBS, 0.5 U/μl RNasin® Plus RNase and 0.1% Triton X100. Next, cells were stained overnight with 0.5 X PFBB in PBS containing 2 U/μl RNasin® Plus RNase inhibitor, 0.1% Triton and 250 ng/μl of the desired intracellular antibody. After staining, the cells were gently washed 6 times with 10 ml 0.1X PFBB in PBS, filtered with 70 um nylon mesh and kept on ice until cell sorting.

Mice

Wild-type (WT) mice (C57Bl/6) were purchased from Harlan and housed in the Weizmann Institute animal facility. Trem^{2-/-} knock-out (KO) mice were kindly provided by Prof. Marco Colonna (Turnbull et al., 2006). Foxp3^{RFP} (Tg(Foxp3-RFP,-cre)) mice were kindly provided by Dr. Jakub Abramsaon. Mice were provided with food and water ad libitum and housed under a strict 12 hr light-dark cycle. Mice were provided with food and water ad libitum and housed under a strict 12 hr light-dark cycle. All experimental procedures were approved by the Institutional Animal Care and Use Committee (IACUC).

Bone Marrow Derived Cell Culture

Female mice were sacrificed by cervical dislocation. To isolate the bone marrow, femora and tibiae from one leg were removed, cleaned from flesh, and flushed with C10 culture medium (RPMI-1640 supplemented with 15% serum, 1%×100 non-essential amino acids, 10 mM HEPES buffer, 1 mM sodium pyruvate, 2 mM L-glutamine, and 50 μM b-mercaptoethanol) using a G21 needle syringe. Flushed bone marrow was filtered through a 70-μm cell strainer and spun down in a cold centrifuge at 300×g for 5 min. Cells were re-suspended in 250 μl RBC lysis solution (Sigma) per leg and incubated for 5 min at room temperature, washed, and resuspended in pre-warmed C10 medium. Cultures were set by plating 2×10⁶ cells in 10 mL C10 supplemented with 20 ng/ml GM-CSF in a 100 mm non-tissue culture plate, and incubated under standard culture conditions (37° C., 5% CO2) (Day 0). On day 2 another 10 ml C10 medium supplemented with 20 ng/ml GM-CSF was added. On day 5, three quarter of the medium was replaced with fresh C10 medium supplemented with 20 ng/ml GM-CSF. On day 7, another 5 ml of C10 medium supplemented with 10 ng/ml GM-CSF. On day 8, non-adherent and loosely adherent cells in the culture supernatant were harvested by gentle washing and re-cultured in a fresh C10 medium supplemented with 10 ng/ml GM-CSF in new non-tissue culture plates and used as starting material for all BMDC experiments.

Tumor Cell Line

MCA-205 fibrosarcoma cell lines were kindly provided by Sergio Quezada group at UCL cancer institute, London, UK. Cells were cultured in DMEM (41965-039) medium supplemented with 10% heat-inactivated FBS, 1 mM sodium pyruvate, 2 mM 1-glutamine, 1% penicillin-streptomycin (Thermo Fisher Scientific). Cells were cultured in 100 mm tissue culture plates in an incubator with humidified air and 5% CO2 at 37° C. Cell lines were validated for lack of mycoplasma infection using primers for mycoplasma-specific 16S rRNA gene region (EZPCR Mycoplasma Kit; Biological Industries).

Tumor Growth Measurements

Mice were inoculated intradermally (i.d.) with 5×10⁵ MCA-205 cells suspended in 100 μl PBS on their right flank. At day 19, tumors volume was measured using a caliper. Tumor volume was assessed by measuring two diameters and calculated using the formula X²×Y×0.52 (where X, smaller diameter and Y, larger diameter).

Isolation of Tumor Infiltrating Leukocytes

Tumor bearing mice were sacrificed at 10 and 19 days after tumor cell inoculation. The tumors underwent mechanical (gentleMACS™ C tube, Miltenyi Biotec Inc., San Diego, Calif.) and enzymatic digestion (0.1 mg/ml DNase type I (Roche), and 1 mg/ml Collagenase IV (Worthington) in RPMI-1640) for 15 min at 37° C. Cells then filtered through 100 μm cell strainer, washed with ice cold sorting buffer, centrifuged (5 min, 4° C., 300 g), and stained with fluorophores conjugated antibodies.

Human Peripheral Blood of Healthy Donors and Isolation of Peripheral Blood PBMC

Blood was taken from 3 healthy peripheral blood donors. The peripheral blood collecting samples is part of the (0220-15-TLV) approval. PBMCs were purified from fresh blood samples by sterile density gradient separation by density centrifugation media (Ficoll-Paque (GE Healthcare Life Sciences)) in a 1:1. Centrifugation (460 g, 25 min,) was performed at 10° C., and the mononuclear cells were carefully aspirated and washed with ice-cold FACS buffer, followed by red blood lysis (Sigma-Aldrich) for 5 min at 4° C. and washing with ice cold FACS buffer.

Isolation of T-Regulatory Cells from Mouse Tumors and Cervical Lymph Nodes

CD45+, TCR-β+, CD11b− and Foxp3+ (by endogenous Foxp3-RFP or anti Foxp3-APC conjugated antibody) cells were isolated from cervical lymph nodes or MCA-205 fiborosarcoma tumors from Foxp3^{RFP} mice.

Flow Cytometry Single Cell Sorting for Mars-Seq 2.0

Following staining, cells were washed and resuspended in cold washing buffer (0.5% BSA and 2 mM EDTA in PBS), stained with fluorophore conjugated anti-mouse CD45 antibody, and filtered through a 70-μm strainer. Before sorting, cells were stained with propidium iodide to exclude dead/dying cells. Cell sorting was performed using a BD FACSAria Fusion flow cytometer (BD Biosciences), gating for CD45+ cells after exclusion of dead cells and doublets. Single cells were sorted into 384-well capture plates containing 2 ml of lysis solution and barcoded poly(T) reverse-transcription (RT) primers for scRNA-seq as described previously (Keren-Shaul et. al 2019). Immediately after sorting, plates were spun down to ensure cell immersion into the lysis solution, snap-frozen on dry ice and stored at 80C until further processing. Cells were analyzed using BD FACSDIVA software (BD Bioscience) and FlowJo software (FlowJo, LLC).

Mass Cytometry (CyToF)

Mouse tumor samples were processed as previously described to achieve single cell suspension. Tumor infiltrating immune cell were enriched using CD45 microbeads (Miltenyi Biotech). Cells were washed with CyTOF PBS and stained with Cisplatin viability stain for 1 min, washed twice and stained with extracellular antibodies cocktail at RT for 30 min. After extracellular staining, cells were washed twice and fixed using the CyTOF Nuclear Antigen Staining Buffer working solution [dilute the 4X Nuclear Antigen Staining Buffer Concentrate (1 part) with Nuclear Antigen Staining Buffer Diluent (3 parts)] for 30 minutes while pipetting every 10 min. Fixed cells were permed by using the CyTOF Perm-S buffer and stained with Intracellular antibodies cocktail for 30 min. Fixed cells were washed twice and resuspended in 4% Formaldehyde (Thermo Fisher) and kept at 4° C. over-night until acquisition day. Stained and Fixed cells were analyzed using the CyTOF 3 (Helios) system (FLUIDIGM). Data was processed using Cytobank.

Flow Cytometry Bulk Cell Sorting for qPCR Experiments:

Cell populations were sorted using BD FACSAria Fusion flow cytometer (BD Biosciences). Prior to sorting, all samples were filtered through a 70-μm nylon mesh. Samples were CD11b+, (Gpnmb+/Pdpn+/Lyc6+) or (Cd11c+ MHCII high/mid). 5,000-10,000 cells were sorted into a low-bind Eppendorf tube containing 40 μl of lysis/binding buffer (Invitrogen). Immediately after sorting, tubes were spun down to ensure cell immersion into the lysis solution, snap frozen on dry ice, and stored at −80° C. until processed.

RT-qPCR for Gene Enrichment Validation:

mRNA from cells sorted in to lysis/binding buffer was captured with 12 μl of Dynabeads oligo(dT) (Invitrogen), washed, and eluted at 85° C. with 10 μl of 10 mM Tris-Cl (pH 7.5). mRNA was reverse transcribed using SuperScript II (ThermoFisher) and cDNA was diluted 1:40 for qPCR measurement using the different genes primers (See Table 3).

TABLE 3

| Primer | Oligonucleotides Sequence | SEQ ID NO: |
|---|---|---|
| Actb_Left | GGAGGGGGTTGAGGTGTT | 58 |
| Actb_Right | TGTGCACTTTTATTGGTCTCAA | 59 |
| | G | |
| Arg1_Left | ATTATCGGAGCGCCTTTCTC | 60 |
| Arg1_Right | AGCAGACCAGCTTTCCTCAG | 61 |
| Trem2_Left | CCCAAGTGGAACACAGCAC | 62 |
| Trem2_Right | GATGCTGGCTGCAAGAAACT | 63 |
| Tnf_Left | CCCTCACACTCAGATCATCTTC | 64 |
| | T | |
| Tnf_Right | GCTACGACGTGGGCTACAG | 65 |
| Cxc12_Left | AAAATCATCCAAAAGATACTG | 66 |
| | AACAA | |
| Cxc12_Right | CTTTGGTTCTTCCGTTGAGG | 67 |
| I11b_Left | ACCCTGCAGTGGTTCGAG | 68 |
| I11b_Right | CTTGCACAAGGAAGCTTGG | 69 |

Droplet-Based scRNA-Seq (10× Chromium)

Fresh or INs-seq-fixed cells were FACS sorted into 0.04% PBS-BSA buffer or INs-seq collection buffer respectively. Cells were stained with trypan blue and counted using light microscopy and then loaded onto a 10× Chromium microfluidics system according to the manufacturer's guidelines. scRNA-seq 5′ gene expression (GEX) libraries were generated using the 10X Genomics Chromium Single Cell 5′ Kit v2 and the 10× Chromium Controller (10× Genomics) according to the 10× Single Cell 5′ v2 protocol guidelines. The 5′ mRNA library was sequenced with Illumina's NextSeq 500 using 75 paired-end reads.

MARS-Seq 2.0 Library Preparation

Single-cell libraries were prepared as previously described (Keren-Shaul et al. 2019). In brief, mRNA from cell sorted into cell capture plates were barcoded and converted into cDNA and pooled using and automated pipeline. The pooled sample is then linearly amplified by T7 in vitro transcription, and the resulting RNA is fragmented and converted into sequencing-ready library by tagging the samples with pool barcodes and illumine sequences during ligation, RT, and PCR. Each pool of cells was tested for library quality and concentration is assessed as described earlier. Overall, barcoding was done on three levels: cell barcodes allow attribution of each sequence read to its cell of origin, thus enabling pooling; unique molecular identifiers (UMIs) allow tagging each original molecule in order to avoid amplification bias; and plate barcodes allow elimination of the batch effect.

mRNA Quality Comparison Between Fixation Methods Using RT-qPCR:

Day 9 culture BMDCs were fixed according to the different fixation protocol guidelines including INS-seq (as described above), and stained for Cd11c. 5000 cells from each protocol were sorted directly into 40 μl of lysis binding buffer (Invitrogen). mRNA was captured with 12 μl of Dynabeads oligo(dT) (Invitrogen) according to protocol. For DSP samples only, mRNA was reverse cross-linked by incubation with 6 mM dNTP, 150 mM Tris pH 8, 90 mM DTT, 0.1% Triton, 6 U/μl RNAsin Plus for 45 minutes at 25° C., followed by 5 minutes at 65° C. and then cooled to 4° C. For all fixation protocols each half of the mRNA material was either reverse transcribed or reverse transcribed and amplified (14 cycles) in the same reaction (RT-PCR). cDNA or amplified cDNA were diluted (1:40) and quantified in qPCR using mouse Actb primers.

Suppression Assay:

Spleen was isolated from 11 weeks WT female (C57Bl/6) mouse and was dissociated into single-cell suspension and filtered through 70 mm cell strainer. Red blood cells were lysed with RBC lysis buffer (Sigma). Splenocytes were passed over CD8 T cell enrichment LS column (Miltenyi). Enriched CD8 T cells were labeled with Cell Proliferation dye eFluor™ 450 (Invitrogen) according to manufacturer's guidelines and co cultured with sterilely sorted intratumoral (MCA205) Cd11b+ Gpnmb+ or Cd11b+ Cxc3r1+ or Cd11b+ Ccr2+ cells separately in a 1:1 ratio. T cells were then activated with CD3/CD28 Dynabeads (Thermo Fisher) according to the kit guidelines. The cells were co-cultured in TC 96 well plat round bottom (Conring) in C10 medium containing recombinant IL-2 (5 ng/ml) and 100 U/mL penicillin/streptomycin. For control, T cells were solo-cultured with or without activation. Cells were harvested after 72 hours, cell suspension was stained with CD8-APC/Cy7 to gate only T cells and T cell proliferation was measured in FACS analysis by Cell Proliferation dye eFluor™ 450 dilution.

Single-Cell RNA-Sequencing Analyses

Single Cell RNA Data Processing (10×)

The Cell Ranger Single Software Suite v.3.1.0 was used to perform sample alignment, demultiplexing and UMI counting using the default parameters. A total of 82,223 single cells consisting of 17 samples (5 Fresh and 12 INs-seq samples) were collected, with the number of cells recovered per samples ranging from 343 to 9507. The mean reads per cells varied from 13,480 and 353,472 with median UMI of 561 to 8092 per cell. Low-quality cells were discarded if the number of expressed genes was smaller than 300. Cells were also removed if their mitochondrial gene expression were larger than 10 percent.

Chromium (10×) Data Integration and Clustering Analysis

For processing of both fresh and INs-seq scRNA-seq data, we used Seurat R package version 3.0. First, we performed filtering of the cells removing cells with less than 300 genes expressed or fraction of mitochondrial gene expression above 10 percent of total UMIs. Next, data for paired fresh and INs-seq samples were normalized using the NormalizeData function and integrated with correction for methods effects across datasets using FindlntegrationAnchors function. We performed Louvain clustering and dimensionality reduction using UMAP algorithm. Marker genes for each cluster have been identified using FindAllMarkers function and Wilcoxon test.

MARS-Seq Processing

scRNA-seq libraries (pooled at equimolar concentration) were sequenced on an Illumina NextSeq 500 at a median sequencing depth of ˜40,000 reads per cell. Sequences were mapped to the mouse (mm10). Demultiplexing and filtering was performed as previously described, with the following adaptations: Mapping of reads was performed using HISAT (version 0.1.6); reads with multiple mapping positions were excluded. Reads were associated with genes if they were mapped to an exon, using the ensembl gene annotation database (embl release 90). Exons of different genes that shared a genomic position on the same strand were considered as a single gene with a concatenated gene symbol. The level of spurious unique molecular identifiers (UMIs) in the data were estimated by using statistics on empty MARS-seq wells, and excluded rare cases with estimated noise >5% (median estimated noise over all experiments was 2%).

Metacell Analysis

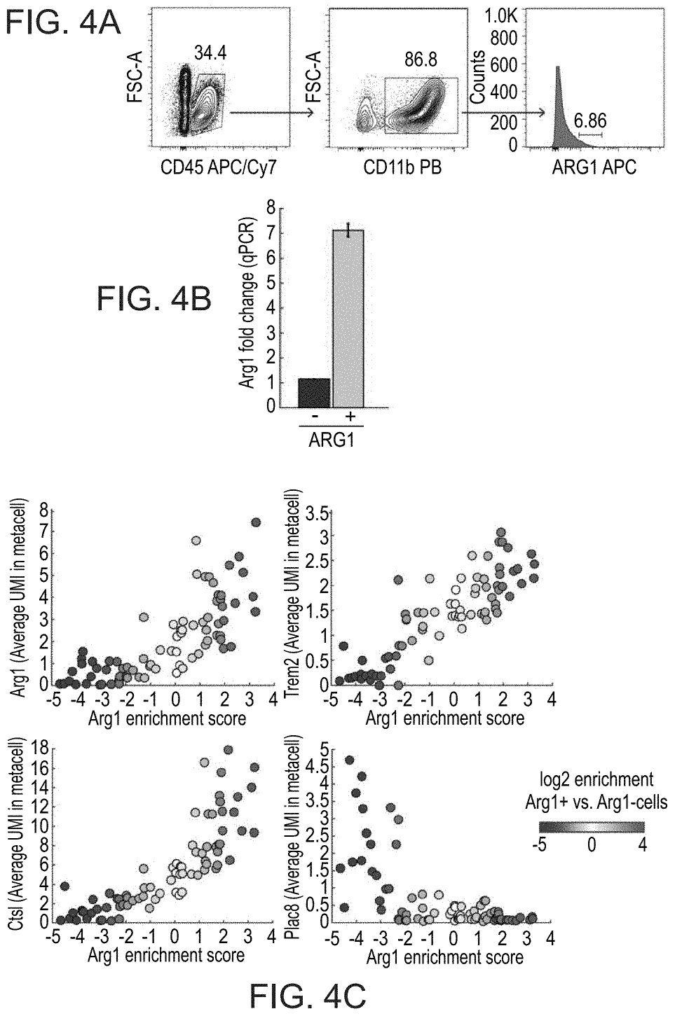

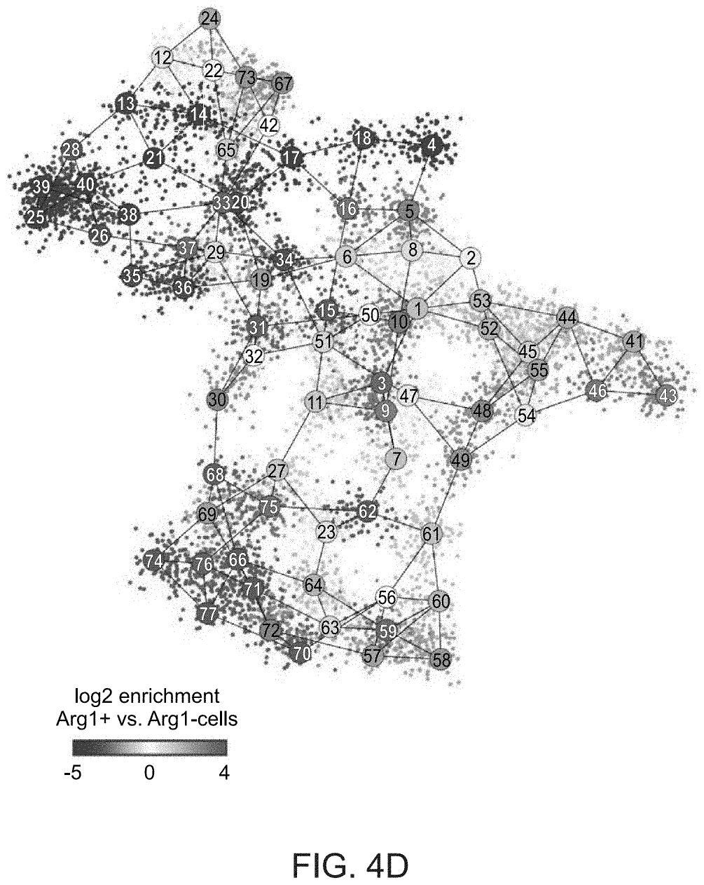

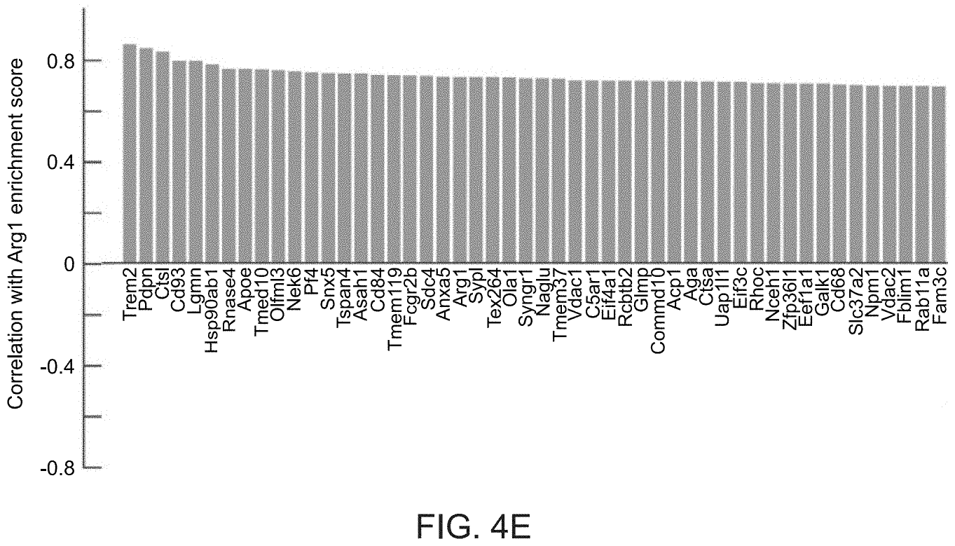

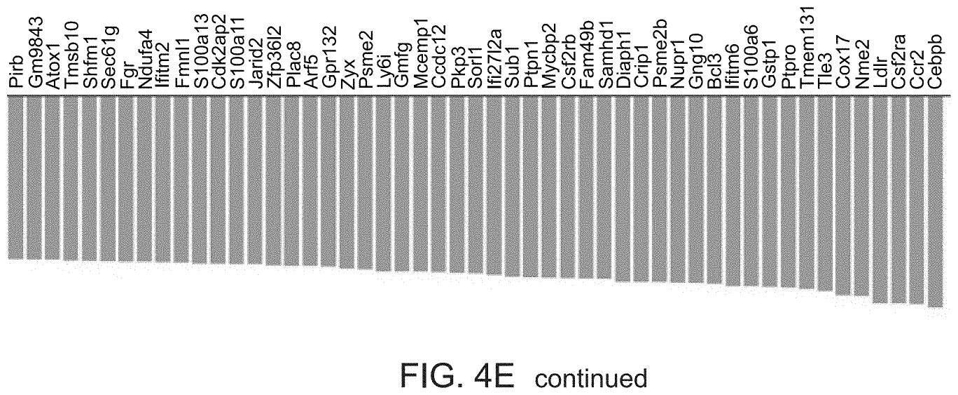

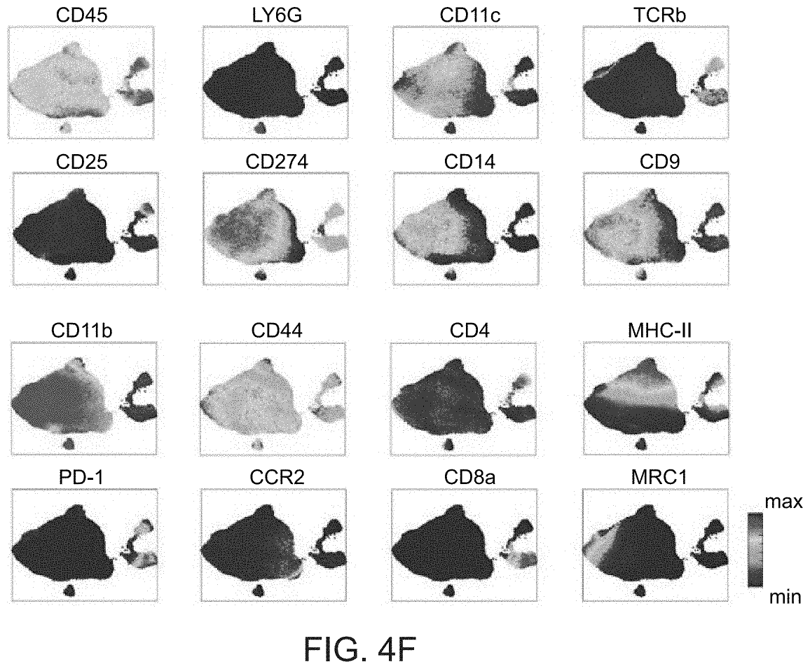

We used the R package “MetaCell” to analyze data from FIGS. 4A-F and FIGS. 5A-C. We removed specific mitochondrial genes, immunoglobulin genes, and genes linked with poorly supported transcriptional models (annotated with the prefix “Rp-”). We then filtered cells with less than 400 UMIs. Gene features were selected using the parameter Tvm=0.3 and a minimum total UMI count >50. We subsequently performed hierarchical clustering of the correlation matrix between those genes (filtering genes with low coverage and computing correlation using a down-sampled UMI matrix) and selected the gene clusters that contained anchor genes. We used K=100, 750 bootstrap iterations and otherwise standard parameters. Metacells were annotated by applying a straightforward analysis of known cell type marker genes (e.g. Ear2, Cx3cr1, Arg1, Trem2, Cd3d, Cd79b, and more). Subsets of Monocytes and Macrophages in FIGS. 5A-C were obtained by hierarchical clustering of the confusion matrix and supervised analysis of enriched genes in homogeneous groups of metacells.

Results

INs-Seq: An Integrated Technology for scRNA-Seq and Intracellular Protein Measurements.

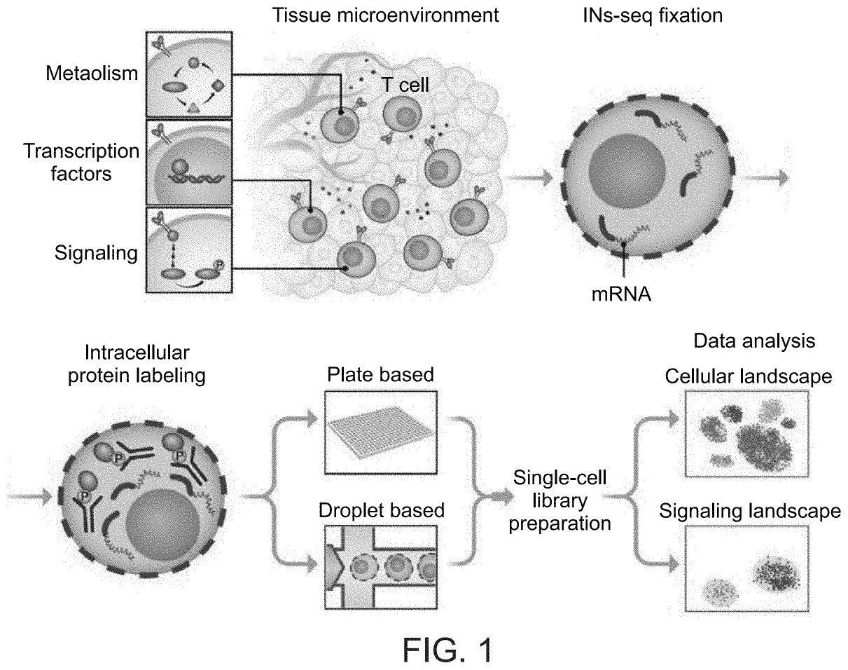

In order to integrate the intracellular signaling state and the cellular transcriptional profile, we developed INs-seq, an integrative technology for intracellular protein immuno-detection followed by scRNA-seq (FIG. 1). In this protocol, cells are fixed and permeabilized using a fixative based on methanol and ammonium sulfate solutions, which precipitates proteins and inhibits enzymatic activity, and enable both RNA preservation and immuno-intracellular staining (STAR methods). The permeabilized cells can then be intracellularly labeled with fluorophore conjugated antibodies and FACS sorted according to their intracellular fluorescent signal intensity, followed by scRNA-seq using plate based or microfluidic based approaches.

Trem2 Defines Two Populations of Tumor Infiltrating Suppressive Myeloid Cells

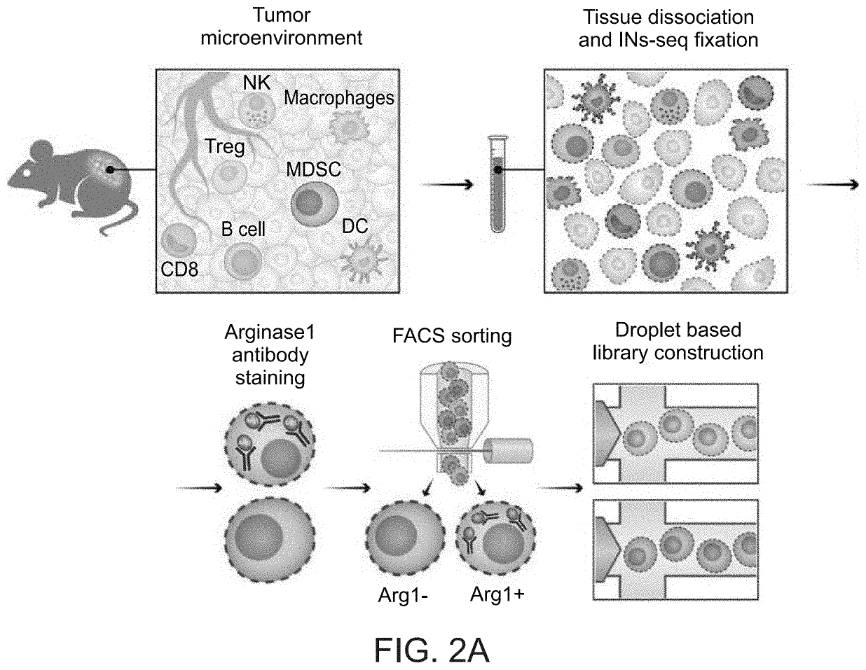

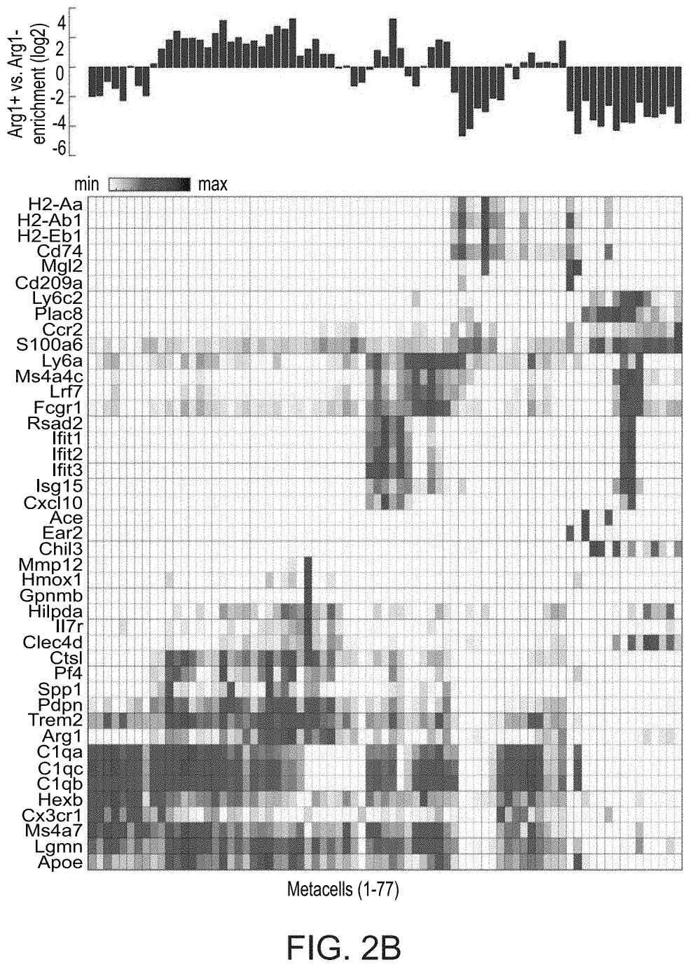

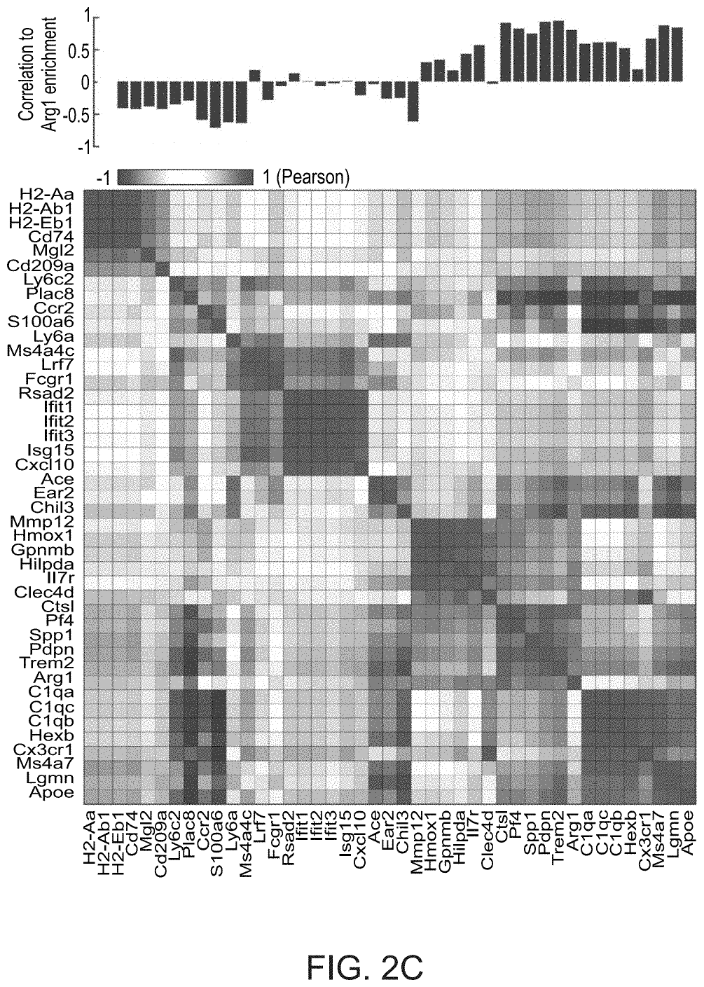

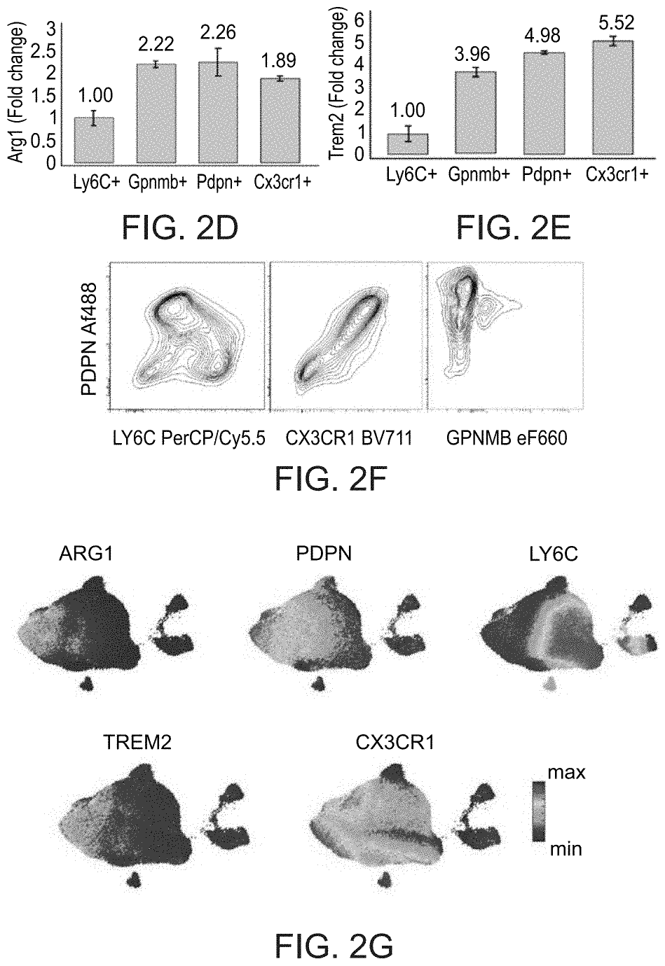

Myeloid cells play a key role in controlling immune activation and suppression. However, to date, there are no clear cell surface molecules, other than broad lineage markers (CD11b, Gr-1), to define myeloid suppressive cells—limiting molecular and functional characterization of this important lineage. The Arginase 1 (Arg1) enzyme, which metabolizes arginine to urea and ornithine, supports many physiological processes such as liver function and collagen production (Caldwell et al., 2018). Within the immune compartment, suppressive myeloid populations activate the Arg1 pathway, depriving the microenvironment from arginine, an essential amino acid for T cells activity (Bronte et al., 2003). Arg1, alongside other metabolic proteins, is a hallmark for tumor associated myeloid suppressor cells that accumulate under pathological conditions (Gabrilovich, 2017; V. Kumar et al., 2016). In order to deeply characterize myeloid suppressor cells within the TME, INs-seq was applied to isolate and profile Arg1 expressing cells from mouse tumor model and define their cellular and molecular pathways (FIG. 2A).

CD45⁺ CD11b⁺ Arg1⁺ and CD45⁺ CD11b⁺ Arg1⁻ cell populations were isolated from the tumor microenvironment (TME) of MCA205 tumor-bearing mice (FIG. 4A). The lymphoid, granulocyte and DC populations were removed from 8,280 QC positive cells for separate analysis. 7,648 cells were defined as monocytes and macrophages based on marker genes expression. Metacell analysis identified 77 metacells comprising 6 distinct populations (FIG. 2B-C). For each metacell we computed an Arg1 enrichment score based on its fraction in the Arg1⁺ over Arg1⁻ populations (FIG. 2B). We found high correlation between Arg1 enrichment score and its transcription levels (FIGS. 4B-D). The myeloid compartment in the TME was characterized by two major Arg1 positive populations; Tumor associated macrophage (TAM), distinguished by the expression of C1qa, Spp1, Cx3Cr1 and Apoe, and a regulatory myeloid cell population (Mreg) expressing Gpnmb, Il7r, Hilpda, Vegfa, Hmox1 and Clec4d, among other differentially expressed genes (FIGS. 2B-C). The four Arg1⁻ populations could be distinguished by expression of specific markers: Plac8, Ly6c2, Ccr2, MHC-II related genes, and a signature associated with type I interferon signaling (FIG. 2B-C). Analysis of the Arg1⁺ and Arg metacells resulted in the de novo identification of a rich set of co-regulated gene modules (FIG. 2C). This analysis further identified Podoplanin (Pdpn) and triggering receptor expressed on myeloid cells 2 (Trem2) as genes significantly correlated with Arg1 expression in both the TAM and Mreg populations (FIGS. 2C and 4C-E). Trem2 is a receptor activated in myeloid cells in several pathologies (Ulland & Colonna, 2018; Zheng et al., 2018) and was shown to have proliferative and survival functions. Moreover, Trem2 was demonstrated to be a key element in disease associated macrophages (DAM) (Keren-Shaul et al., 2017) and Lipid-Associated Macrophages (LAM) (Jaitin et al., 2019) regulating lipid metabolism, phagocytosis and immune suppression. In order to validate these findings, the present inventors used the surface markers identified in the single cell data to enrich for the myeloid suppressive cells expressing high Arg1 protein, and then measured their Arg1 and Trem2 mRNA quantity using qPCR. Antibodies targeting Pdpn, Cx3cr1 and Gpnmb were used as they define distinct markers for the two major Arg1^{high} subpopulations, while Ly6C⁺ cells were highly correlated with the Arg1^{low} subsets. Consistent with this data, qPCR analysis of Pdpn, Gpnmb and Cx3cr1 populations, detected higher expression levels of Arg1 and Trem2 transcripts as compared to the Ly6C⁺ population (FIGS. 2D-E).

To further validate these results, the present inventors analyzed CD45⁺ CD11b⁺ cells from MCA205 mouse tumors stained for same cell surface markers depicting the different Arg1⁺ and Arg1⁻ intra-tumoral myeloid populations within the TME. They validated that Pdpn^{high} and Ly6C^{high} subsets were clearly detectable as separate populations by flow cytometry. Furthermore, the Pdpn^{high} myeloid population was strongly associated with Cx3cr1^{high}, and Gpmnb^{high} markers (FIG. 2F). In order to further devise a sorting strategy and characterize the Arg1⁺ and Arg1⁻ intra-tumoral myeloid populations, CyToF Mass-Cytometry was used to profile the intra-tumoral immune populations of MCA205. UMAP analysis of a large set of proteins defined by the single cell data, revealed similar results to those observed in the FACS analysis (FIG. 2G). The CyToF results further confirmed the transcriptional findings, defining two distinct myeloid populations marked by Pdpn and Ly6C. In line with the single cell data, the Pdpn population overlapped with expression of Cx3cr1, Trem2, Arg1 and CD206 (Mrc1) (FIGS. 2G and 4F). In summary, INs-seq analysis of Arg1 expression defined the molecular characteristics of two distinct myeloid populations that share the expression of Arg1 and the Trem2 receptor; a tumor associated macrophage population and a regulatory myeloid cell population.

Trem2 Promotes T Cell Dysfunction and Tumor-Immune Escape

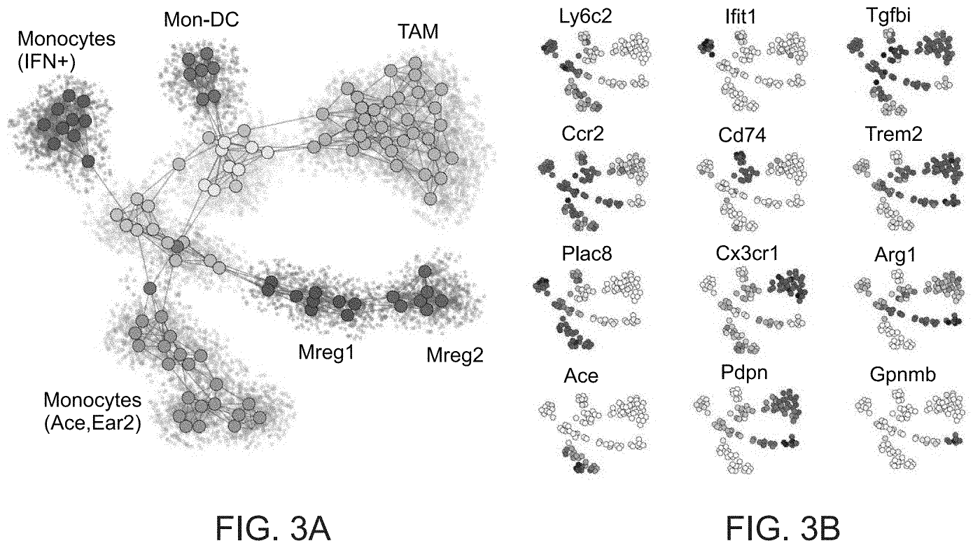

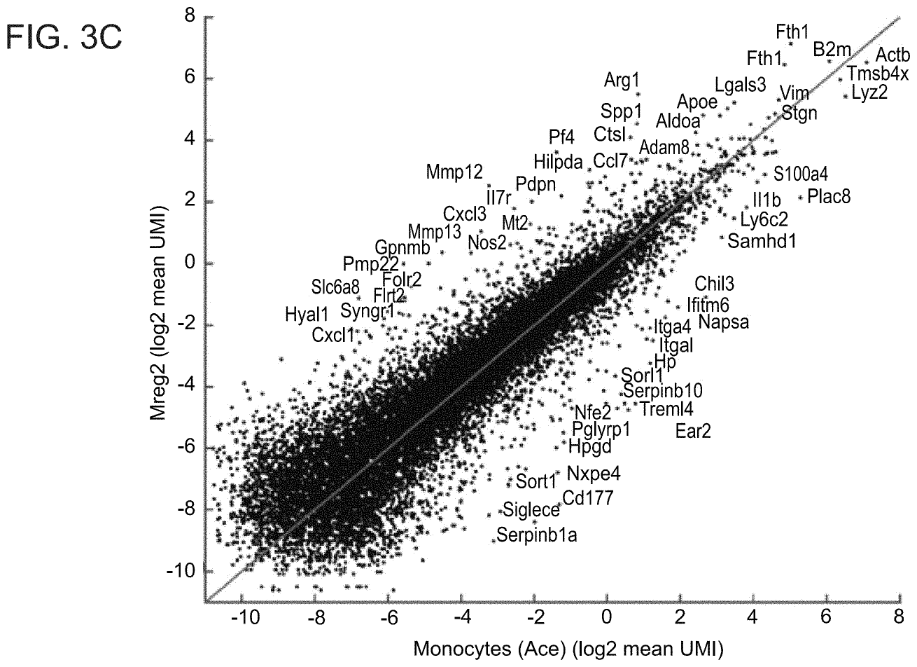

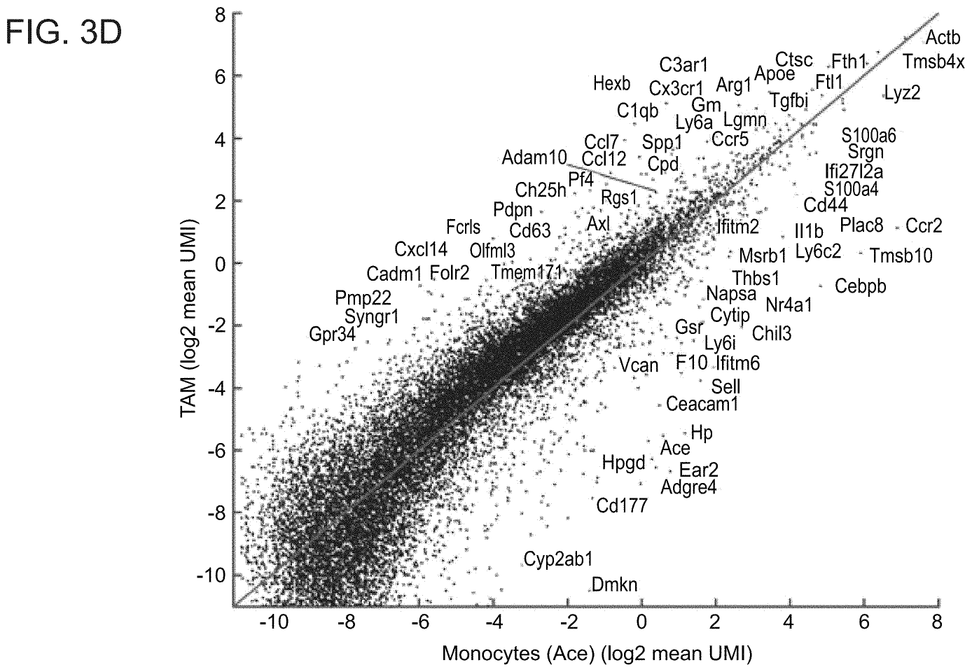

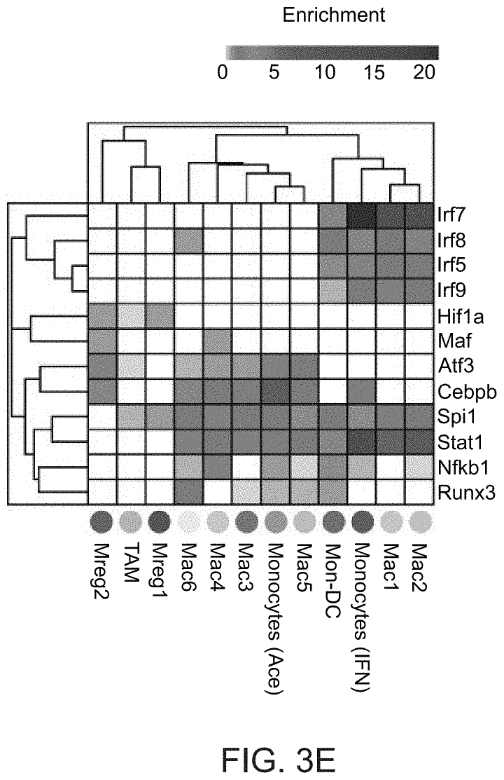

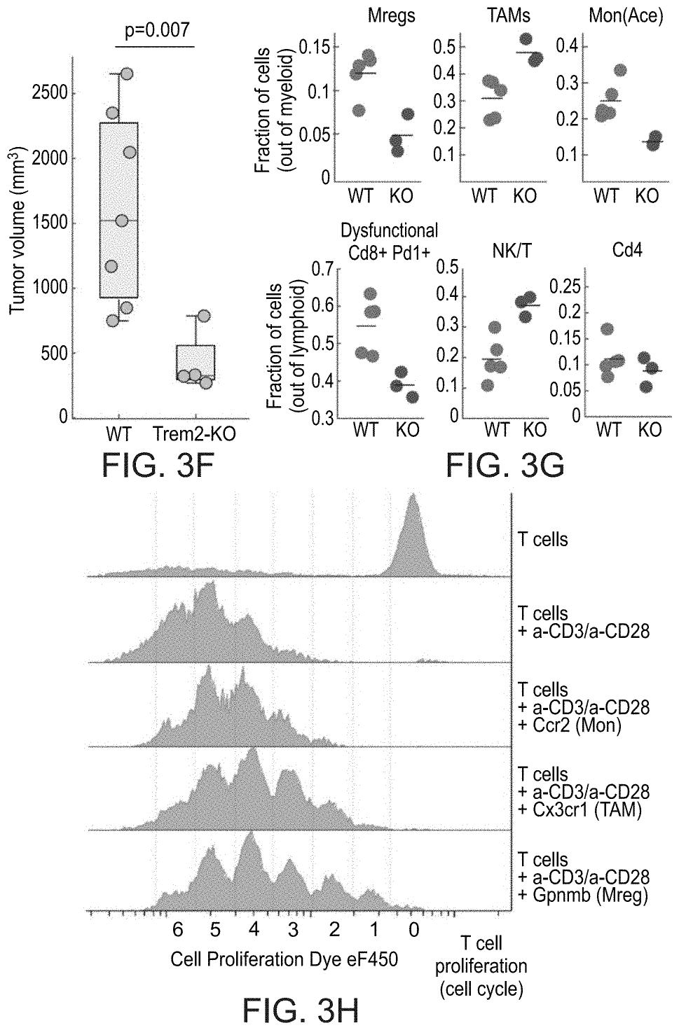

To confirm the IN s-seq tumor map and gain deeper molecular characterization of the tumor associated myeloid populations, immune cells (CD45⁺) were sorted from MCA205 tumors for MARS-seq analysis. The MetaCell algorithm was used to identify homogeneous groups of cells from single-cell RNA-seq data, resulting in a map of 115 metacells (FIG. 3A). The lymphoid, granulocytes and DC populations were removed from the myeloid map and analyzed them independently (FIG. 5A). Similar to the INs-seq tumor map, Arg1 expression was highly correlated with Trem2 and Pdpn expression, while Ly6C, Ccr2 and Plac8 represented Arg1 myeloid populations (FIG. 3B). Consistent with this analysis, the Arg1⁺ Trem2⁺ populations can be subdivided into two distinct programs: TAM s, characterized by mature macrophages markers, such as Cx3cr1, Apoe and C1qa, and Mreg, which are monocytic-like cells expressing Gpnmb, Il7r and several hypoxia related genes such as Hilpda, Hmox1 and Vegfa (FIGS. 3B-D). Multiple TFs were found that correlated with the myeloid suppressive programs, including known regulators and several TFs that have not been previously associated with suppressive myeloid cells (FIG. 3E). The present inventors trained a lasso-regularized cross validated linear model (STAR methods), predicting the different programs with high accuracy based on expression of TFs only (FIGS. 3E and 5B). Maf, Cebpb, Atf3 and Hifla were identified as potential Mreg regulators and Spil and Hifla for TAMs. The monocytes cluster enriched with type I interferon signaling showed TF enrichment for Stat1, Irf9 and Irf7.

In order to better understand the regulatory mechanisms of Arg1⁺ cells, the present inventors further analyzed the single-cell data, seeking potential regulators that may perturb the accumulation of the suppressive myeloid cells. Among them, the present inventors identified Trem2 as a promising target. Trem2 is highly correlated with Arg1⁺ myeloid cells and has been shown to promote myeloid cell proliferation, survival and immune suppression in various pathologies (Gervois & Lambrichts, 2019; Zhong et al., 2017), Trem2 was also shown to be expressed in myeloid cells in human tumors and its deficiency in mouse tumor model abrogated tumor growth (Tang et al., 2019; Zhang et al., 2018). Comparison between MCA205 tumor growth in Trem2+/+ (WT) and Trem2−/− mice showed a significant reduction in tumor volume in the Trem2−/− mice (FIGS. 3F and 5C). To further study the role of Trem2 on the intra-tumoral immune regulation, the present inventors performed scRNA-seq of immune cells derived from MCA205 bearing mice, from Trem2+/+ and Trem2−/− background, capturing a total of 15,808 QC positive cells. Focusing on the Trem2 expressing myeloid cells, the present inventors found significant reduction in the Mreg population in the TME, together with an increase in the TAM population, in the Trem2−/− mice compared to WT (FIG. 3G). Examination of the lymphoid compartment in WT and Trem2−/− mice revealed a notable decrease of CD8⁺ dysfunctional T cells (expressing PD1 and Tim3), along with a significant expansion of the NK and cytotoxic T cell populations (FIG. 3G). To assess the functional impact of the different tumor infiltrating myeloid populations on activated T lymphocytes proliferation, a T cell proliferation assay was used. Tumor infiltrating myeloid populations (Mreg, TAMs and Ccr2⁺) were isolated, FACS sorted, and co-cultured with Cell proliferation dye-labeled splenic isolated naïve CD8 T cells, activated with α-CD3 and α-CD28. While CD8⁺ T cells co-cultured with intra-tumoral Ccr2⁺ monocytes showed a full proliferative behavior with no indication of suppression, we observed a considerable reduction in proliferation of activated CD8⁺ T cells that were co-cultured with Cx3cr1⁺ TAM cells and an even more dominant suppression phenotype when cultured with Gpnmb⁺ Mreg cells (FIG. 3H). Altogether the results demonstrate that Arg1⁺ tumor infiltrating myeloid cells contain two molecularly distinct myeloid populations; TAM and Mreg. Trem2 knockout mice show that while both populations are defined by Trem2 expression, only the Mreg populations are affected by Trem2 ablation. Importantly, Trem2 deficiency leads to significant reduction in Mreg, dysfunctional CD8⁺ T cells and tumor growth.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of reducing the immune suppressor activity of myeloid cells, the method comprising contacting myeloid cells with an effective amount of an agent which specifically reduces the amount and/or activity of myeloid cells expressing both Triggering Receptor Expressed On Myeloid Cells 2 (Trem2) and Transmembrane glycoprotein NMB (Gpnmb), thereby reducing the immune suppressor activity of myeloid cells.

2. The method of claim 1, wherein said contacting is in vivo.

3. The method of claim 1, wherein said contacting is ex vivo.

4. A method of treating cancer in a subject in need thereof, the method comprising administering to the subject an effective amount of an agent which specifically downregulates the amount and/or the activity of myeloid cells of the subject expressing both Triggering Receptor Expressed On Myeloid Cells 2 (Trem2) and Transmembrane glycoprotein NMB (Gpnmb), thereby treating the cancer.

5. A method of treating cancer in a subject in need thereof, the method comprising:
(a) reducing the immune suppressor activity of myeloid cells according to the method of claim 3, wherein said myeloid cells are derived from the subject; and subsequently
(b) transplanting said myeloid cells to the subject, thereby treating the cancer.

6. The method of claim 1, wherein said agent comprises a bi-specific antibody.

7. The method of claim 6, wherein a first target of said bi-specific antibody is Trem2 and a second target of said bi-specific antibody is Gpnmb.

8. The method of claim 4, wherein said first agent and said second agent are inhibitory antibodies.

9. The method of claim 6, wherein said bi-specific antibody is attached to a cytotoxic agent.

10. The method of claim 4, wherein said cancer is a solid cancer.

11. The method of claim 10, wherein said solid cancer is selected from the group consisting of lung cancer, liver cancer, ovarian cancer, gastric cancer and breast cancer.

12. The method of claim 1, further comprising administering to the subject a therapeutically effective amount of a checkpoint inhibitor.

13. The method of claim 1, further comprising administering to the subject a therapeutically effective amount of a Brutons tyrosine kinase (Btk) inhibitor.

14. A bispecific antibody, comprising a first antigen-binding domain capable of specifically binding to Trem2 and a second antigen-binding domain capable of specifically binding to Gpnmb.

15. A method of treating cancer in a subject comprising:
(a) analyzing in a sample of the subject for the presence of myeloid cells which express both Trem2 and Gpnmb; and
(b) when there is an amount of said myeloid cells above a predetermined amount, treating the subject with a therapeutically effective amount of an agent that targets Trem2 and/or Gpnmb; or when there is an amount of said cells below a predetermined amount treating the subject with a therapeutically effective amount of a chemotherapeutic agent other than said agent that targets Trem2 and/or Gpnmb.

16. The method of claim 15, wherein said agent that targets Trem2 and/or Gpnmb antibody is an antibody optionally attached to a cytotoxic agent.

17. The method of claim 15, further comprising administering to the subject a therapeutically effective amount of a checkpoint inhibitor.
